SEQUENCE LISTING (46 sequences)

SEQ ID NO: 1 - DNA, 22 bp, Homo sapiens
  source 1..22: mol_type = genomic DNA
tcggattcggggagaggtttgg

SEQ ID NO: 2 - DNA, 25 bp, Homo sapiens
  source 1..25: mol_type = genomic DNA
tcagtggacagtagacggaggaaag

SEQ ID NO: 3 - DNA, 22 bp, Homo sapiens
  source 1..22: mol_type = genomic DNA
gagaaggaggtagtgctgctgg

SEQ ID NO: 4 - DNA, 24 bp, Homo sapiens
  source 1..24: mol_type = genomic DNA
gatgcacaaggtaatgtctcggta

SEQ ID NO: 5 - DNA, 24 bp, Homo sapiens
  source 1..24: mol_type = genomic DNA
ggtggagctacggacaatgaatgg

SEQ ID NO: 6 - DNA, 23 bp, Homo sapiens
  source 1..23: mol_type = genomic DNA
gcttgaggcactgactgagactg

SEQ ID NO: 7 - DNA, 23 bp, Homo sapiens
  source 1..23: mol_type = genomic DNA
tgttggtcctgctggcaagaatg

SEQ ID NO: 8 - DNA, 23 bp, Homo sapiens
  source 1..23: mol_type = genomic DNA
gtcaccttgttcgcctgtctcac

SEQ ID NO: 9 - DNA, 21 bp, Homo sapiens
  source 1..21: mol_type = genomic DNA
tgattctacccacggcaagtt

SEQ ID NO: 10 - DNA, 21 bp, Homo sapiens
  source 1..21: mol_type = genomic DNA
tgatgggtttcccattgatga

SEQ ID NO: 11 - DNA, 25 bp, Homo sapiens
  source 1..25: mol_type = genomic DNA
agcagcgactctgaagaagaacaag

SEQ ID NO: 12 - DNA, 22 bp, Homo sapiens
  source 1..22: mol_type = genomic DNA
ggatgaccctgactcggacctc

SEQ ID NO: 13 - DNA, 23 bp, Homo sapiens
  source 1..23: mol_type = genomic DNA
cattgctggtccagtctgcttcg

SEQ ID NO: 14 - DNA, 24 bp, Homo sapiens
  source 1..24: mol_type = genomic DNA
tggtgaatgacagtgcggttatgg

SEQ ID NO: 15 - DNA, 23 bp, Homo sapiens
  source 1..23: mol_type = genomic DNA
aggcgtggttcaactaggagagg

SEQ ID NO: 16 - DNA, 23 bp, Homo sapiens
  source 1..23: mol_type = genomic DNA
ccgcagaggtggtattggttgtg

SEQ ID NO: 17 - DNA, 24 bp, Homo sapiens
  source 1..24: mol_type = genomic DNA
acccaccaccaacattcaggaaac

SEQ ID NO: 18 - DNA, 25 bp, Homo sapiens
  source 1..25: mol_type = genomic DNA
ctcaggactcttcaccaagcacttc

SEQ ID NO: 19 - DNA, 22 bp, Homo sapiens
  source 1..22: mol_type = genomic DNA
ttcggcacggtccaacaattcc

SEQ ID NO: 20 - DNA, 25 bp, Homo sapiens
  source 1..25: mol_type = genomic DNA
ttgaaactacgccatctccagaagc

SEQ ID NO: 21 - DNA, 24 bp, Homo sapiens
  source 1..24: mol_type = genomic DNA
tcaaaccagcagcagcagatacag

SEQ ID NO: 22 - DNA, 25 bp, Homo sapiens
  source 1..25: mol_type = genomic DNA
tccgtattgcctgccgaaataactc

SEQ ID NO: 23 - DNA, 25 bp, Homo sapiens
  source 1..25: mol_type = genomic DNA
atgtcgtccatcttgccattcactc

SEQ ID NO: 24 - DNA, 22 bp, Homo sapiens
  source 1..22: mol_type = genomic DNA
tgttctccaccacctgctcctc

SEQ ID NO: 25 - DNA, 24 bp, Homo sapiens
  source 1..24: mol_type = genomic DNA
tcctggctacctgagtgaagatgg

SEQ ID NO: 26 - DNA, 22 bp, Homo sapiens
  source 1..22: mol_type = genomic DNA
tcggattcggggagaggtttgg

SEQ ID NO: 27 - DNA, 23 bp, Homo sapiens
  source 1..23: mol_type = genomic DNA
ggagcagcaagagcaaggagaag

SEQ ID NO: 28 - DNA, 25 bp, Homo sapiens
  source 1..25: mol_type = genomic DNA
tcagtggacagtagacggaggaaag

SEQ ID NO: 29 - DNA, 25 bp, Homo sapiens
  source 1..25: mol_type = genomic DNA
ctgccgtctgtgagagtcatattgg

SEQ ID NO: 30 - DNA, 25 bp, Homo sapiens
  source 1..25: mol_type = genomic DNA
caagagcgagatgtgatgtctggtc

SEQ ID NO: 31 - DNA, 24 bp, Homo sapiens
  source 1..24: mol_type = genomic DNA
cctgcttgctctccttgtcttctg

SEQ ID NO: 32 - DNA, 25 bp, Homo sapiens
  source 1..25: mol_type = genomic DNA
actgctccaacttcccttcactttg

SEQ ID NO: 33 - DNA, 23 bp, Homo sapiens
  source 1..23: mol_type = genomic DNA
tgttggtcctgctggcaagaatg

SEQ ID NO: 34 - DNA, 23 bp, Homo sapiens
  source 1..23: mol_type = genomic DNA
gtcaccttgttcgcctgtctcac

SEQ ID NO: 35 - DNA, 23 bp, Homo sapiens
  source 1..23: mol_type = genomic DNA
gctgttgatagaccaccgcttcc

SEQ ID NO: 36 - DNA, 21 bp, Homo sapiens
  source 1..21: mol_type = genomic DNA
tgccctgttcctctgccttcc

SEQ ID NO: 37 - DNA, 23 bp, Homo sapiens
  source 1..23: mol_type = genomic DNA
cttagagggctacgggacggatg

SEQ ID NO: 38 - DNA, 25 bp, Homo sapiens
  source 1..25: mol_type = genomic DNA
tcgtggttggttctgtaatgcttcc

SEQ ID NO: 39 - DNA, 25 bp, Homo sapiens
  source 1..25: mol_type = genomic DNA
cacaggtcgctactatgccatgaag

SEQ ID NO: 40 - DNA, 25 bp, Homo sapiens
  source 1..25: mol_type = genomic DNA
cacaggtcgctactatgccatgaag

SEQ ID NO: 41 - DNA, 24 bp, Homo sapiens
  source 1..24: mol_type = genomic DNA
aagcgtcaagccaaacacaaacag

SEQ ID NO: 42 - DNA, 25 bp, Homo sapiens
  source 1..25: mol_type = genomic DNA
ccccacatcactgaagtccacatac

SEQ ID NO: 43 - DNA, 25 bp, Homo sapiens
  source 1..25: mol_type = genomic DNA
acctaagcaacttgtccagcatcag

SEQ ID NO: 44 - DNA, 22 bp, Homo sapiens
  source 1..22: mol_type = genomic DNA
ggatcggtcaaggttggcagtg

SEQ ID NO: 45 - DNA, 24 bp, Homo sapiens
  source 1..24: mol_type = genomic DNA
ggtggagctacggacaatgaatgg

SEQ ID NO: 46 - DNA, 23 bp, Homo sapiens
  source 1..23: mol_type = genomic DNA
gcttgaggcactgactgagactg

(19) United States
(12) Patent Application Publication
(10) Pub. No.: US 2024/0245825 A1
(43) Pub. Date: Jul. 25, 2024

(54) MULTI-LAYERED SCAFFOLD FOR OSTEOCHONDRAL COMPLEX DEFECT REPAIR

(71) Applicant: THE HONG KONG POLYTECHNIC UNIVERSITY, Hong Kong (CN)

(72) Inventors: Xin ZHAO, Hong Kong (CN); Tianpeng XU, Hong Kong (CN); Yuhe YANG, Hong Kong (CN)

(21) Appl. No.: 18/417,184

(22) Filed: Jan. 19, 2024

Related U.S. Application Data
(60) Provisional application No. 63/480,698, filed on Jan. 20, 2023.

(51) Int. Cl. A61L 24/00; A61K 38/18; A61L 24/02; A61L 24/04

(52) U.S. Cl. CPC A61L 24/0015; A61K 38/1841; A61K 38/1875; A61L 24/02; A61L 24/043; A61L 2300/414; A61L 2400/06; A61L 2430/06

(57) ABSTRACT

A multi-layered scaffold useful for osteochondral complex defects. The multi-layered scaffold can be prepared by photopolymerizing a multi-layer scaffold precursor including: a first layer disposed on a surface of a second layer, wherein the first layer includes a first tri-block poly(lactide-co-propylene glycol-co-lactide) dimethacrylate; and the second layer includes a second tri-block poly(lactide-co-propylene glycol-co-lactide) dimethacrylate, hydroxyapatite nanoparticles covalently conjugated to one or more hydroxyethyl methacrylate moieties via an optional linker, and a photoinitiator, wherein the nanoparticles comprise hydroxyapatite, tricalcium phosphate, silicon dioxide, bioglass, or a mixture thereof.

### CROSS-REFERENCE TO RELATED APPLICATIONS

The present application claims priority from U.S. Provisional Patent Application No. 63/480,698, filed on Jan. 20, 2023, which is hereby incorporated by reference in its entirety.

### REFERENCE TO SEQUENCE LISTING

The Sequence Listing identified as Sequence_Listing_P24912US01.xml; Size: 40.2 kilobytes; and Date of Creation: Jan. 16, 2024, filed herewith, is hereby incorporated by reference in its entirety.

### TECHNICAL FIELD

The present disclosure relates to a subaqueously crosslinkable multi-layered scaffold precursor and a multi-layered scaffold prepared therefrom useful for repairing osteochondral complex defects.

### BACKGROUND

Osteochondral (OC) complex is comprised of two connective but distinct zones: the cartilage, and the subchondral bone; with varied biological lineage in terms of mechanical properties, extracellular matrix (ECM) compositions, and cellular phenotypes.^{[1]} Current methods in treating OC defects involve premade bi-layered scaffolds that mimic the zonal organization characteristics of the natural OC regions. Despite their routine use, the premade implants are invasive, with potential local tissue damage and postoperative complications, long recovery times, and patient discomfort.^{[2]} As an alternative, injectable adhesives exhibit great superiority over prefabricated scaffolds thanks to their minimal invasiveness, accessibility to deep-seated areas, and high plasticity for morphing into undefined damaged geometry.^{[3]} Hydrogels such as collagen, gelatin, and alginate have been widely applied as injectable scaffolds given their excellent injectability, biocompatibility, and capacity to load biomolecules (e.g., growth factors).^{[4]} Unfortunately, their high hydrophilicity raises concerns for scaffold integrity as their fluidity and lack of clinging mechanisms render them at risk of injection spillage and incomplete filling of the defect site. Together with their poor resistance to possible hydraulic pressure during surgery, dislocation under in vivo dynamic conditions (e.g., shear stress), and steep release of biomolecules, these drawbacks complicate the use of subaqueously crosslinked hydrogels.^{[5]} Scientists thus turn to liquid hydrophobic materials, including poly(methyl methacrylate), poly(propylene fumarate), poly(glycerol sebacate) fumarate, polyanhydrides, etc.^{[6]} When these materials are fabricated into bi-layered structures in situ, they, however, undergo lengthy (up to few hours) and highly exothermic (˜90° C.) crosslinking reaction, compromising their biocompatibility and capability to carry heat-labile bioactive molecules; nonetheless, the product structures often suffer from weak interconnection and delamination due to the absence of strong interfacial bonding^{[7]} Ultimately, an injectable hydrophobic material that can crosslink subaqueously and rapidly, form biomimicking bi-layered structures with robust interlayer bonding, and allow for long-term release of growth factors, is highly sought after for OC repair.

There is thus a need for improved scaffolds for OC defect repair that overcomes at least some of the disadvantages in the art.

### SUMMARY

During OC reconstruction, careful manipulation of material mechanical properties contributes to surgical useability and native modulation of regenerative cells. Significant effort was spent on optimizing the mechanical properties of biomaterials to induce osteogenic and chondrogenic differentiation of mesenchymal stem cells (MSCs).^{[8]} Generally, biophysical and biochemical cues were reported to play a crucial role in directing the commitment process. Extracellular matrix (ECM) stiffness, which matches that of native tissues (e.g., cartilage (1-10 MPa)^{[9]} and bone (100-1,000 MPa),^{[10]} was reported to modulate MSC differentiation into corresponding chondrocytes and osteoblasts through specific integrin and focal adhesion pathways,^{[11]} whereas biochemical cues, such as growth factors, serve a vital role in modulating the OC microenvironment and stimulating cartilage and bone repair through various signaling pathways.^{[12]} Therefore, we speculate the smart manipulation of the crosstalk between the biophysical and biochemical cues in bi-layered OC scaffolds could be an effective strategy to synergically regulate the cell fates and enhance OC repair.

Provided herein is an injectable hydrophobic adhesive that can rapidly crosslink in a subaqueous environment, form bi-layered structures with strong interlayer bonding and synergically modulate the biophysical and biochemical cues for enhancing OC repair (FIG. 1). A bi-layered structure is constructed using liquid hydrophobic tri-block poly (lactide-co-propylene glycol-co-lactide) dimethacrylate (PₘLₙDMA), wherein m and n represent the unit length of propylene glycol (PPG) and lactide (LA), respectively. PₘLₙDMA is adopted as the OC complex's cartilage phase, and a polymer encapsulating methacrylated hydroxyapatite nanoparticles (PₘLₙDMA/MH) as the mineralized subchondral bone phase. To match the biophysical properties of native tissues, systemically modulation of the PₘLₙDMA composition (m and n) and the ratio of MH to PₘLₙDMA enables control over the mechanical properties of both phases. Given the hydrophobic nature of the materials, the two phases can be injected and freely conform to the irregular geometry of the OC defect, omitting the concern of material dispersal among the body fluid under an aqueous environment. Furthermore, olefins present in the PₘLₙDMA and the PₘLₙDMA/MH layers quickly form strong covalent bonding at their interface via photo-induced crosslinking into a bi-layered scaffold structure. The crosslinking reaction generates relatively low heat (<37° C.) and hence, can be used as a vehicle for carrying growth factor TGF-β1 in PₘLₙDMA and BMP-2 in PₘLₙDMA/MH. It was also found that the adhesives modulate the cartilage regeneration through synergistically activating the TGF-β/Smad and Hippo-YAP/TAZ signaling pathways, while enhancing the bone regeneration through synergistically activation of the FAK and PI3K-Akt signaling pathways. The development of the injectable hydrophobic laminous PₘLₙDMA/MH adhesive described herein addresses current limitations in OC repair and coordinates mechano-biochemical microenvironments of the defect sites for facilitating OC repair.

In a first aspect, provided herein is a multi-layered scaffold comprising a cartilage phase layer disposed on a surface of a bone phase layer, wherein the multi-layered scaffold is prepared by photopolymerizing a multi-layer scaffold precursor comprising: a first layer disposed on a surface of a second layer, wherein the first layer comprises a first tri-block poly(lactide-co-propylene glycol-co-lactide) dimethacrylate; and the second layer comprises a second tri-block poly(lactide-co-propylene glycol-co-lactide) dimethacrylate, nanoparticles covalently conjugated to one or more hydroxyethyl methacrylate moieties via an optional linker, and a photoinitiator, wherein the nanoparticles comprise hydroxyapatite, tricalcium phosphate, silicon dioxide, bioglass, or a mixture thereof.

In certain embodiments, each of the first tri-block poly(lactide-co-propylene glycol-co-lactide) dimethacrylate and the second tri-block poly(lactide-co-propylene glycol-co-lactide) dimethacrylate independently comprises polypropylene glycolide having the structure —[O(CHMe)(CH₂)]ₘO—, wherein m is 2-40.

In certain embodiments, each of the first tri-block poly(lactide-co-propylene glycol-co-lactide) dimethacrylate and the second tri-block poly(lactide-co-propylene glycol-co-lactide) dimethacrylate independently comprise polylactide and polypropylene glycolide in a molar ratio between 1-5 to 1, respectively.

In certain embodiments, the second layer comprises hydroxyapatite nanoparticles covalently conjugated to one or more hydroxyethyl methacrylate moieties via an optional linker at a concentration of 10-70% wt/wt relative to the total weight of the second tri-block poly(lactide-co-propylene glycol-co-lactide) dimethacrylate and hydroxyapatite nanoparticles covalently conjugated to one or more hydroxyethyl methacrylate moieties via an optional linker.

In certain embodiments, each of first layer and the second layer independently further comprises one or more bioactive factors.

In certain embodiments, the one or more bioactive factors are selected from the group consisting of a nucleic acid, a protein, a peptide, a cytokine, a hormone, a cell, and a growth factor.

In certain embodiments, the first layer further comprises TGF-β1; and the second layer further comprises BMP-2

In certain embodiments, the hydroxyapatite nanoparticles are covalently conjugated to one or more hydroxyethyl methacrylate moieties via a diisocyanate linker.

In certain embodiments, the diisocyanate linker is OCN(CH₂)ₙNCO, wherein n is a whole number selected from 2-8.

In certain embodiments, the photoinitiator is photoinitiator is an acylphosphine oxide.

In certain embodiments, at least one exterior surface of the multi-layered scaffold comprises microgrooves, micropillars, or a combination thereof.

In certain embodiments, an exterior surface of the cartilage phase layer comprises microgrooves and an exterior surface of the bone phase layer comprises micropillars.

In certain embodiments, the step of photopolymerizing comprises photopolymerizing the first layer and the second layer simultaneously or photopolymerizing the second layer and then photopolymerizing the first layer.

In certain embodiments, each of the first tri-block poly(lactide-co-propylene glycol-co-lactide) dimethacrylate and the second tri-block poly(lactide-co-propylene glycol-co-lactide) dimethacrylate independently comprises polypropylene glycolide having the structure —[O(CHMe)(CH₂)]ₘO—, wherein m is 7-34; each of the first tri-block poly(lactide-co-propylene glycol-co-lactide) dimethacrylate and the second tri-block poly(lactide-co-propylene glycol-co-lactide) dimethacrylate independently comprise polylactide and polypropylene glycolide in a molar ratio between 2-4 to 1, respectively; the hydroxyapatite nanoparticles are covalently conjugated to one or more hydroxyethyl methacrylate moieties via a diisocyanate having the structure: OCN(CH₂)ₙNCO, wherein n is a whole number selected from 4-8; the second layer comprises the hydroxyapatite nanoparticles covalently conjugated to one or more hydroxyethyl methacrylate moieties via the diisocyanate linker at a concentration of 10-50% wt/wt relative to the total weight of the second tri-block poly(lactide-co-propylene glycol-co-lactide) dimethacrylate and the hydroxyapatite nanoparticles covalently conjugated to one or more hydroxyethyl methacrylate moieties via an optional linker; and each of the first layer and the second layer optionally independently further comprises one or more bioactive factors.

In certain embodiments, each of the first tri-block poly(lactide-co-propylene glycol-co-lactide) dimethacrylate and the second tri-block poly(lactide-co-propylene glycol-co-lactide) dimethacrylate independently comprises polypropylene glycolide having the structure —[O(CHMe)(CH₂)]ₘO—, wherein m is 7; each of the first tri-block poly(lactide-co-propylene glycol-co-lactide) dimethacrylate and the second tri-block poly(lactide-co-propylene glycol-co-lactide) dimethacrylate independently comprise polylactide and polypropylene glycolide in a molar ratio of 2 to 1, respectively; the hydroxyapatite nanoparticles are covalently conjugated to one or more hydroxyethyl methacrylate moieties via a diisocyanate linker having the structure: OCN(CH₂)₆NCO; and the second layer comprises the hydroxyapatite nanoparticles covalently conjugated to one or more hydroxyethyl methacrylate moieties via the diisocyanate linker at a concentration of 50% wt/wt relative to the total weight of the second tri-block poly(lactide-co-propylene glycol-co-lactide) dimethacrylate and the hydroxyapatite nanoparticles covalently conjugated to one or more hydroxyethyl methacrylate moieties via the diisocyanate linker.

In certain embodiments, the first layer further comprises TGF-β1; and the second layer further comprises BMP-2.

In a second aspect, provided herein is a method of fabricating the multi-layer scaffolded described herein, the method comprising: providing a first layer precursor; providing a second layer precursor; and photopolymerizing the first layer precursor and the second layer precursor thereby forming the multi-layer scaffolded, wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof.

In a third aspect, provided herein is a method of fabricating the multi-layer scaffolded described herein, the method comprising: depositing a second layer precursor on a substrate thereby forming the second layer; depositing a first layer precursor on a surface of the second layer thereby forming the multi-layer scaffold precursor; and photopolymerizing the multi-layer scaffold precursor thereby forming the multi-layer scaffolded, wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof.

In a fourth aspect, provided herein is a method of repairing an osteochondral complex (OC) defect in a subject in need thereof, the method comprising: depositing a second layer precursor at the OC defect site thereby forming a second layer; depositing a first layer precursor on a surface of the second layer thereby forming the multi-layer scaffold precursor; and photopolymerizing the multi-layer scaffold precursor thereby forming the multi-layer scaffolded described herein, wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof.

### BRIEF DESCRIPTION OF THE DRAWINGS

The above and other objects and features of the present disclosure will become apparent from the following description of the disclosure, when taken in conjunction with the accompanying drawings.

FIG. 1. Schematics showing the underwater-crosslinking laminous adhesives with strong interlayer bonding and mechanochemical cues for OC repair. The adhesives direct the commitment of mesenchymal stem cells into corresponding chondrocytes and osteoblasts and facilitate the OC repair by mediating the crosstalk between the mechanotransduction and growth factor mediated signaling pathways. PₘLₙDMA=poly(lactide-co-propylene glycol-co-lactide) dimethylacrylates, ‘m’ refers to the unit length of propylene glycol and ‘n’ refers to the molar ratio of lactide; MH=hydroxyethyl methacrylate hydroxyapatite; BMP-2 morphogenetic protein-2; TGF-β1=transforming growth factor beta-1; YAP=yes-associated protein.

FIG. 2. Characterization of injectable adhesives. (A) Schematics showing the structure and application of the PₘLₙDMA and PₘLₙDMA/MH adhesives for OC repair. (B) Polymerization ratio with time of PₘLₙDMA and PₘLₙDMA/MH detected by FTIR. (C) Exothermic temperature profiles of PₘLₙDMA and PₘLₙDMA/MH during photocrosslinking; (D) Compressive versus tensile modulus for PₘLₙDMA and PₘLₙDMA/MH compared with the human cartilage and bone. (E and F) Viscosity evaluation of adhesives with different compositions of PₘLₙDMA and PₘLₙDMA/MH; (Gi-vi) Underwater injectability and crosslinking ability of the adhesives. (Hi-iv) Plasticity of the bi-layered scaffolds in accommodating molds of different shapes. (Ii-iv) SEM images of the bonding interface, PₘLₙDMA, and PₘLₙDMA/MH layer of the scaffold. (J and K) The bonding strength of different adhesives under normal and shearing force. (L) Cumulative release of TGF-β1 and BMP-2 from P₇L₂DMA and P₇L₂DMA/50MH for 60 days detected by ELISA. D and W means samples photo-crosslinked under dry (D) or wet (W) conditions. 7/4, 7/2, 17/4, and 34/4 refer to the P₇L₄DMA, P₇L₂DMA, P₁₇L₄DMA and P₃₄L₄DMA groups respectively. 10MH, 30MH and 50MH refer to 10, 30 and 50 w/w % methacrylated hydroxyapatite nanoparticles encapsulated in the adhesives. Sample size n=3 for all experiments, one-way ANOVA with a Tukey's post hoc test was used for multiple comparisons. Data are presented as mean±SD.

FIG. 3. Chondrogenesis and osteogenesis potential of the adhesives. (A and B) Safranin 0 and alcian blue staining of rBMSCs. (C and D) ALP and ARS staining of rBMSCs. (E and F) Quantification of Safranin 0 and alcian staining. (G and H) Quantification of ALP activity and ARS staining. 7/2, 7/2/50MH, 7/2@TGF-β1 and 7/2/50MH@BMP-2 refer to P₇L₂DMA, P₇L₂DMA/50MH, TGF-β1 in P₇L₂DMA and BMP-2 in P₇L₂DMA/50MH group, respectively. Sample size n=3 for all experiments by a two-way ANOVA with a Tukey's post hoc test for multiple comparisons. Data are presented as mean±SD. *p<0.05 and ^{#}p<0.05 are considered significantly different compared with the 7/2/50MH group and the 7/2 group respectively.

FIG. 4. Bioinformatic study of laminous adhesives on modulating rBMSC's chondrogenic differentiation. (A) PCA plot shows the obvious separation gene expression of rBMSCs on different adhesives. (B) Venn diagram of differentially expressed genes treated by 7/2@TGF-β1 and 7/2/50MH. (C) Volcano map showing differential gene expressions (DGEs). (D) KEGG enrichment scatter plot. (E) Gene expression heatmap. (F) Quantitative real-time polymerase chain reaction (qRT-PCR) analysis results. (G) Schematic showing the synergistic effect of 7/2@TGF-β1 adhesive in stimulating Integrin-Hippo-YAP/TAZ and TGF/Smad signaling pathway for promoting chondrogenic differentiation in rBMSCs.

FIG. 5. Bioinformatic study of laminous adhesives on modulating rBMSC's osteogenic differentiation. (A) PCA plot shows the obvious separation gene expression of rBMSCs on different adhesives. (B) Venn diagram of differentially expressed genes treated by 7/2/50MH@BMP-2 and 7/2. (C) Volcano map showing differential gene expressions (DGEs). (D) KEGG enrichment scatter plot. (E) Gene expression heatmap. (F) Quantitative real-time polymerase chain reaction (qRT-PCR) analysis results. (G) Schematic showing the synergetic effect of 7/2/50MH@BMP-2 adhesives in stimulating the PI3K-Akt-RUNX2 signaling pathway for promoting osteogenic differentiation in rBMSCs.

FIG. 6. The in vivo osteochondral (OC) regeneration efficacy of laminous adhesives in a rabbit OC defect model. (A) Gross appearance of the repaired cartilage at 8 and 12 weeks. (B) Micro-CT scans of the repaired osteochondral tissues (origin indicating the regenerated subchondral bone). (C) ICRS score of the repaired OC tissues; (D and E) Quantification of the BMD and BV/TV. Sample size n=6 for all experiments by a two-way ANOVA with a Tukey's post hoc test for multiple comparisons. Data presented as mean±SD. *p<0.05 and ^{#}p<0.05 are considered significantly different compared with the blank and the 7/2 group respectively.

FIG. 7. Histology analysis showing the in vivo OC regeneration efficacy of laminous adhesives in a rabbit OC defect model. (A and B) The Safranin-O/Fast-Green and Masson trichrome staining of the repaired OC tissues. (C) Histological scores of the repaired OC tissues. (D) Quantification of the red staining areas in Masson trichomes staining. Sample size n=6 for all experiments by a two-way ANOVA with a Tukey's post hoc test for multiple comparisons. Data are presented as mean±SD. *p<0.05 and ^{#}p<0.05 are considered significantly different compared with the 7/2 group and the 7/2/50MH group respectively.

FIG. 8. FTIR spectra of synthesized PₘLₙDMA

FIG. 9. ¹H NMR spectra of synthesized PₘLₙDMA

FIG. 10. FTIR spectra of synthesized methacrylated hydroxyapatite (MH) nanoparticles.

FIG. 11. TGA plots of hydroxyapatite nanoparticles (HA) and methacrylated hydroxyapatite nanoparticles (MH) at 5° C./min.

FIG. 12. (A-D) Polymerization kinetics of PₘLₙDMA and PₘLₙDMA/MH detected by FTIR.

FIG. 13. (A-D) Exothermic temperature profiles of PₘLₙDMA and PₘLₙDMA/MH during photocrosslinking.

FIG. 14. Viscosity evaluation of adhesives with different compositions of (A) P₇L₄DMA/MH, (B) P₁₇L₄DMA/MH and (C) P₃₄L₄DMA/MH.

FIG. 15. (A-B) The mechanical properties of 7/2 and 7/2/50MH injectable adhesive in wet and dry crosslinking environment.

FIG. 16. (i-ii) The deformation of bi-layered injectable adhesives under different kinds of forces.

FIG. 17. The water contact angle of P₇L₂DMA and P₇L₂DMA/50MH. Sample size n=3 for all experiments. Student's t-test was used for multiple comparisons. Data are presented as mean±SD. *p<0.05 is considered significantly different compared with the P₇L₂DMA group.

FIGS. 18. (A and B) Immunofluorescence staining of collagen-1 and collagen-2 with the rBMSCs cultured with the osteogenic/chondrogenic medium (blank), adhesives' extract medium and the blank medium supplemented with growth factors (BMP-2 or TGF-β1); (C) Quantification of the relative immunofluorescence intensity. Sample size n=3 for all experiments. One-way ANOVA with a Tukey's post hoc test was used for multiple comparison. Data are presented as mean±SD. *p<0.05 is considered significantly different compared with the blank group.

FIG. 19. (A) Morphology of rBMSCs cultured on different adhesives; (B) Quantification of cell spreading area. Sample size n=3 for all experiments by a one-way ANOVA with a Tukey's post hoc test for multiple comparisons. Data are presented as mean±SD. *p<0.05 and ^{#}p<0.05 are considered significantly different compared with the 7/2 group and the 7/2@ TGF-β1 group respectively.

FIG. 20. (A) Cell viability of rBMSCs cultured on different adhesives; (B and C) Quantification of cell viability and proliferation. Sample size n=3 for all experiments by a two-way ANOVA with a Tukey's post hoc test for multiple comparisons. Data are presented as mean±SD. *p<0.05 is considered significantly different compared with the 7/2 group.

FIG. 21. (A) Aggrecan staining of the rBMSCs seeded on different adhesives on day 7; (B) Relative fluorescence intensity of the Aggrecan staining. Sample size n=3 for all experiments by a one-way ANOVA with a Tukey's post hoc test for multiple comparisons. Data are presented as mean±SD. *p<0.05 and ^{#}p<0.05 are considered significantly different compared with the 7/2/50MH group and the 7/2 group respectively.

FIG. 22. mRNA expression of (A) Collagen-1 and (B) Aggrecan of the rBMSCs seeded on different adhesives on day 3 and 7. Sample size n=3 for all experiments by a two-way ANOVA with a Tukey's post hoc test for multiple comparisons. Data are presented as mean±SD. *p<0.05 and ^{#}p<0.05 are considered significantly different compared with the 7/2/50MH group and the 7/2 group, respectively.

FIG. 23. (A) OCN staining of the rBMSCs seeded on different adhesives on day 7; (B) Relative fluorescence intensity of the OCN staining. Sample size n=3 for all experiments by a one-way ANOVA with a Tukey's post hoc test for multiple comparisons. Data are presented as mean±SD. *p<0.05 and ^{#}p<0.05 are considered significantly different compared with the 7/2 group and the 7/2/50MH group respectively.

FIG. 24. mRNA expression of (A) RUNX2 and (B) Collagen-1 of the rBMSCs seeded on different adhesives on day 3 and 7. Sample size n=3 for all experiments by a two-way ANOVA with a Tukey's post hoc test for multiple comparisons. Data are presented as mean±SD. *p<0.05 and ^{#}p<0.05 are considered significantly different compared with the 7/2 group and the 7/2/50MH group respectively.

FIG. 25. Pearson correlation evaluation between different samples.

FIG. 26. GO classification of (A) up-regulated and (B) down-regulated differentially expressed genes of rBMSCs (7/2@TGF-β1 vs 7/2/50MH).

FIG. 27. Down-regulated enriched KEGG pathways of rBMSCs (7/2@TGF-β1 vs 7/2/50MH).

FIG. 28. GO classification of (A) up-regulated and (B) down-regulated differentially expressed genes of rBMSCs (7/2/50MH@BMP-2 vs 7/2).

FIG. 29. Down-regulated enriched KEGG pathways of rBMSCs (7/2/50MH@BMP-2 vs 7/2).

FIG. 30. Table 1 PₘLₙDMA synthesis efficiency. “N_{LA}”/“E_{LA}” and “N_{MA}”/“E_{MA}” represent the average number/synthesis efficiency for LA and methacrylate attached to the PPG chain, respectively.

FIG. 31. Table 2 Mechanical properties of PₘLₙDMA and PₘLₙDMA/MH.

FIG. 32. Table 3 The primer sequences used for qRT-PCR.

FIG. 33. Table 4 Primer sequences for genes selected for RNA-seq validation.

FIG. 34. Fabrication and characterization of the adhesive Janus periosteum. (A) Schematic diagram depicting the photo-crosslinking-assisted injection molding technique to fabricate the Janus periosteum. SEM images of the adhesive Janus periosteum including microgroove-patterned surface with different microgroove width of (B) 40 μm, (C) 80 μm and (D) 120 μm and (E) fibrillar arrays on the adhesion surface. (F) XPS spectra and (G) N 1s XPS spectra of the naked PₘLₙDMA and poly(dopamine methacrylamide-co-hydroxyethyl methacrylate) (PDMH)-coated PₘLₙDMA with varying PDMH concentrations. The PₘLₙDMA, PₘLₙDMA/DMA-1, PₘLₙDMA/DMA-3, and PₘLₙDMA/DMA-5 denoted the naked Janus periosteum and the Janus periosteum with 1, 3, 5 mg/mL PDMH coating respectively. The magnified image is the high-resolution N1s XPS spectra of the coatings.

FIG. 35. SEM images of cross-section of the adhesive Janus periosteum

FIG. 36. Characterization of the adhesion strength of the Janus periosteum. (A) Schematic diagram showing the testing protocol of the shear and normal adhesion strength. (B) Application of the anisotropic periosteum to fresh bone, which could withstand an 800 g weight. (C) Quantification of the shear (Ci) and normal (Cii) adhesion strength under dry condition. (D) Quantification of the shear (Di) and normal (Dii) adhesion strength under wet condition. GP referred to the naked gecko inspired adhesion surface, PDMH-1 referred to the no pattern surface with 1 mg/mL PDMH coating, and the GP-PDMH-1, GP-PDMH-3, and GP-PDMH-5 referred to the GP surface with 1, 3, and 5 mg/mL PDMH coating respectively. *p<0.05 and ^{#}p<0.05 were compared with the no pattern and GP group respectively.

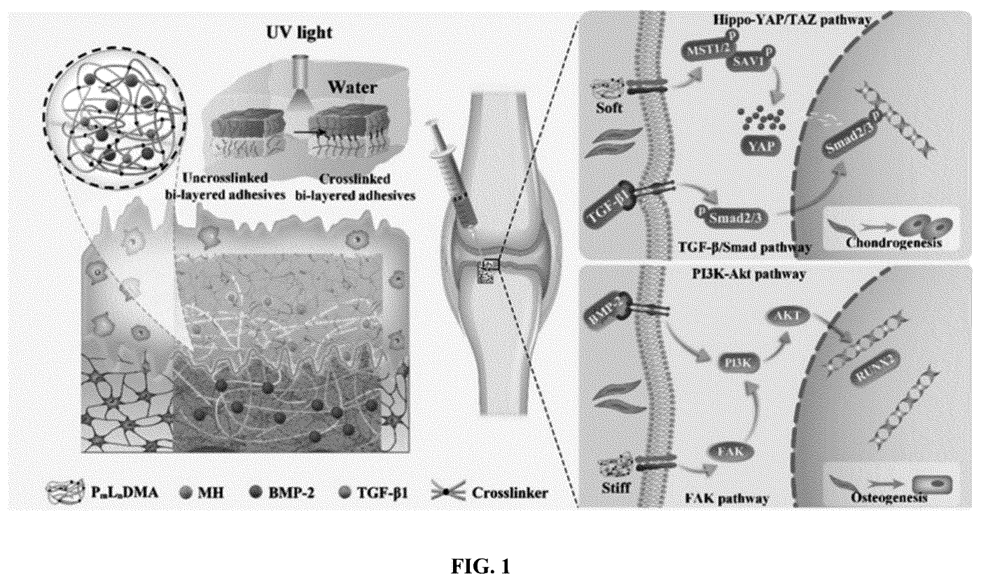

### DETAILED DESCRIPTION

### Definitions

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

As used herein, a “polymeric compound” (or “polymer”) refers to a molecule including a plurality of one or more repeating units connected by covalent chemical bonds. A polymeric compound can be represented by General Formula I:

$${}^{*} - {( - {( Ma )}_{x} — {( Mb )}_{y} — )}_{z}^{*} \qquad General Formula I$$

wherein each Ma and Mb is a repeating unit or monomer. The polymeric compound can have only one type of repeating unit as well as two or more types of different repeating units. When a polymeric compound has only one type of repeating unit, it can be referred to as a homopolymer. When a polymeric compound has two or more types of different repeating units, the term “copolymer” or “copolymeric compound” can be used instead. For example, a copolymeric compound can include repeating units where Ma and Mb represent two different repeating units. Unless specified otherwise, the assembly of the repeating units in the copolymer can be head-to-tail, head-to-head, or tail-to-tail. In addition, unless specified otherwise, the copolymer can be a random copolymer, an alternating copolymer, or a block copolymer. For example, General Formula I can be used to represent a copolymer of Ma and Mb having x mole fraction of Ma and y mole fraction of Mb in the copolymer, where the manner in which comonomers Ma and Mb is repeated can be alternating, random, regiorandom, regioregular, or in blocks, with up to z comonomers present. In addition to its composition, a polymeric compound can be further characterized by its degree of polymerization (n) and molar mass (e.g., number average molecular weight (M) and/or weight average molecular weight (Mw) depending on the measuring technique(s)). The polymers described herein can exist in numerous stereochemical configurations, such as isotactic, syndiotactic, atactic, or a combination thereof.

As used herein, the term “subject” refers to any animal (e.g., a mammal), including, but not limited to, humans, non-human primates, canines, felines, and rodents.

The present disclosure provides a multi-layered scaffold comprising a cartilage phase layer disposed on a surface of a bone phase layer, wherein the multi-layered scaffold is prepared by photopolymerizing a multi-layer scaffold precursor comprising: a first layer disposed on a surface of a second layer, wherein the first layer comprises a first tri-block poly(lactide-co-propylene glycol-co-lactide) dimethacrylate; and the second layer comprises a second tri-block poly(lactide-co-propylene glycol-co-lactide) dimethacrylate, nanoparticles covalently conjugated to one or more hydroxyethyl methacrylate moieties via an optional linker, and a photoinitiator, wherein the nanoparticles comprise hydroxyapatite, tricalcium phosphate, silicon dioxide, bioglass, or a mixture thereof. The first tri-block poly(lactide-co-propylene glycol-co-lactide) dimethacrylate and the second tri-block poly(lactide-co-propylene glycol-co-lactide) dimethacrylate can be the same or different.

Photopolymerization of the multi-layer scaffold precursor can result in crosslinking of the first tri-block poly(lactide-co-propylene glycol-co-lactide) dimethacrylate, crosslinking between the second tri-block poly(lactide-co-propylene glycol-co-lactide) dimethacrylate and the hydroxyethyl methacrylate functionalized hydroxyapatite nanoparticles; and crosslinking between the first tri-block poly(lactide-co-propylene glycol-co-lactide) dimethacrylate and the second tri-block poly(lactide-co-propylene glycol-co-lactide) dimethacrylate.

The step of photopolymerization can comprise irradiating the second layer and the first layer sequentially and/or simultaneously (i.e., irradiating the multi-layer scaffold precursor) with electromagnetic radiation (e.g., ultraviolet light).

Each of the first tri-block poly(lactide-co-propylene glycol-co-lactide) dimethacrylate and the second tri-block poly(lactide-co-propylene glycol-co-lactide) dimethacrylate can independently comprise polypropylene glycolide blocks having the structure —[O(CHMe)(CH₂)]ₘO—, wherein m is 1-40, 1-35, 1-30, 1-25, 1-20, 1-15, 1-10, 2-10, 3-10, 4-10, 5-10, 6-10, 6-8, 5-9, 4-10, 3-11, 7-34, 17-34, or 7-17. In certain embodiments, m is about 7.

The molar ratio of the polylactide blocks to the polypropylene glycolide in each of the first tri-block poly(lactide-co-propylene glycol-co-lactide) dimethacrylate and the second tri-block poly(lactide-co-propylene glycol-co-lactide) dimethacrylate can range from 1-10 to 1, 1-9 to 1, 1-8 to 1, 1-7 to 1, 1-6 to 1, 2-6 to 1, 3-6 to 1, 3-5 to 1, respectively. In certain embodiments, the molar ratio of the polylactide blocks to the polypropylene glycolide in each of the first tri-block poly(lactide-co-propylene glycol-co-lactide) dimethacrylate and the second tri-block poly(lactide-co-propylene glycol-co-lactide) dimethacrylate is about 4 to about 1.

Advantageously, the tensile modulus and the compressive modulus of the cartilage phase layer described herein is similar to human cartilage when it is prepared from a first tri-block poly(lactide-co-propylene glycol-co-lactide) dimethacrylate comprises glycolide blocks having the structure —[O(CHMe)(CH₂)]ₘO—, wherein m is about 7 and comprises a molar ratio of the polylactide blocks to the polypropylene glycolide of about 4 to about 1, respectively.

Advantageously, the tensile modulus and the compressive modulus of the cartilage phase layer described herein is similar to human cartilage when it is prepared from a first tri-block poly(lactide-co-propylene glycol-co-lactide) dimethacrylate comprises glycolide blocks having the structure —[O(CHMe)(CH₂)]ₘO—, wherein m is about 7 and comprises a molar ratio of the polylactide blocks to the polypropylene glycolide of about 4 to about 1 or about 2 to about 1, respectively.

In certain embodiments, the first layer further comprises hydroxyethyl methacrylate. The hydroxyethyl methacrylate can be present in the first layer at a concentration of 1-15% wt/wt, 1-10% wt/wt, 5-15% wt/wt, or 5-10% wt/wt. In certain embodiments, the first layer further comprises hydroxyethyl methacrylate at a concentration of about 9% wt/wt. The nanoparticles can comprise hydroxyapatite, tricalcium phosphate, silicon dioxide, bioglass, or a mixture thereof. In certain embodiments, the nanoparticles comprise hydroxyapatite.

The second layer can comprise the nanoparticles covalently conjugated to one or more hydroxyethyl methacrylate moieties via an optional linker at a concentration of 1-90% wt/wt, 10-90% wt/wt, 20-90% wt/wt, 30-90% wt/wt, 40-90% wt/wt, 50-90% wt/wt, 60-90% wt/wt, 70-90% wt/wt, 80-90% wt/wt, 10-80% wt/wt, 10-70% wt/wt, 10-60% wt/wt, 10-50% wt/wt, 20-50% wt/wt, 30-50% wt/wt, 40-50% wt/wt, 30-60% wt/wt, 40-60% wt/wt, or 45-55% wt/wt, relative to the total weight of the second tri-block poly(lactide-co-propylene glycol-co-lactide) dimethacrylate and the nanoparticles covalently conjugated to one or more hydroxyethyl methacrylate moieties via an optional linker. In certain embodiments, the second layer comprises the nanoparticles covalently conjugated to one or more hydroxyethyl methacrylate moieties via an optional linker at a concentration of about 50% wt/wt the relative to the total weight of the second tri-block poly(lactide-co-propylene glycol-co-lactide) dimethacrylate and the hydroxyapatite nanoparticles covalently conjugated to one or more hydroxyethyl methacrylate moieties via an optional linker.

Advantageously, the tensile modulus and the compressive modulus of the bone phase layer described herein is similar to human bone when it is prepared from a second tri-block poly(lactide-co-propylene glycol-co-lactide) dimethacrylate comprises glycolide blocks having the structure —[O(CHMe)(CH₂)]ₘO—, wherein m is about 7 and comprises a molar ratio of the polylactide blocks to the polypropylene glycolide of about 4 to about 1 or about 2 to about 1, respectively; and the second layer comprises the hydroxyapatite nanoparticles covalently conjugated to one or more hydroxyethyl methacrylate moieties via an optional linker at a concentration of about 50% wt/wt the relative to the total weight of the second tri-block poly(lactide-co-propylene glycol-co-lactide) dimethacrylate and the hydroxyapatite nanoparticles covalently conjugated to one or more hydroxyethyl methacrylate moieties via an optional linker.

The photoinitiator is not particularly limited and can be any photoinitiator that is substantially non-toxic to cells or organisms within acceptable tolerances, including substantially non-carcinogenic and substantially non-immunogenic in a subject. Exemplary photoinitiators include 1-hydroxycyclohexylphenyl ketone (e.g., Irgacure® 184 available from Ciba Specialty Chemical (Hawthorne, N.Y.), bis (2,6-dimethoxybenzoyl)-2,4,4-trimethylpentyl phosphine oxide (e.g., commercial blends Irgacure® 1800, 1850, and 1700 available from Ciba Specialty Chemical), 2,2-dimethoxyl-2-phenyl acetophenone (e.g., Irgacure 651, available from Ciba Specialty Chemical), bis(2,4,6-trimethyl benzoyl)phenyl-phosphine oxide (Irgacure® 819), (2,4,6-trimethylbenzoyl)diphenyl phosphine oxide (Lucerin TPO, available from BASF (Munich, Germany)), ethoxy (2,4,6-trimethylbenzoyl)phenyl phosphine oxide (Lucerin TPO-L from BASF), and combinations thereof. In certain embodiments, the photoinitiator is an acylphosphine oxide selected from the group consisting of TPO, TPO-Na, BAPO (Irgacure® 819), BAPO-ONa, BAPO-Oli, and LAP. In certain embodiments, the photoinitiator is a water-soluble photoinitiator selected from the group consisting of Irgacure® 2959, LAP, BAPO-OLi, BAPO-ONa, TPO-Na, MAPO-3, VA-086, Eosin-Y, camphor-quinone, riboflavin, WSPI, BDEA, P2CK, Lithium phenyl (2,4,6-trimethylbenzoyl) phosphinate (LAP) and the like.

In alternative embodiments, the photoinitiator is present in both the first layer and second layer or present in the first layer, but not the second layer.

The present disclosure contemplates all linkers capable of reacting with the nucleophiles present on the surface of the nanoparticles and the alcohol of hydroxyethyl methacrylate. In certain embodiments, the linker is a diisocyanate or a siloxane comprising an isocyanate or blocked isocyanate. In certain embodiments, the siloxane has the formula OCN(CH₂)ₚSi(OR)₃, wherein p is 1-6 and R is methyl or ethyl. The diisocyanate can be a C₂-C₁₂ alkyl diisocyanate. In certain embodiments, the diisocyanate is OCN(CH₂)ₙNCO, wherein n is a whole number selected from 2-12, 2-10, 2-8, 2-6, 4-8, 4-6, or 5-6. In certain embodiments, diisocyanate is hexamethylene diisocyanate (m=6).

In certain embodiments, the hydroxyapatite nanoparticles are further covalently conjugated to one or more dopamine methacrylamide moieties via an optional linker, wherein the optional linker is as described herein.

The nanoparticles can have an average diameter of 20-500 nm, 20-400 nm, 20-300 nm, 20-200 nm, 20-100 nm, 50-100 nm, or 80-100 nm.

Advantageously, the multi-layered scaffold is capable of slow release of bioactive factors present in the first layer and/or second layer. In certain embodiments, each of the first layer and the second layer optionally further comprises one or more bioactive factors selected from the group consisting of an antibiotic, a hormone, an analgesic, an anti-inflammatory agent, a growth factor, an angiogenic factor, a chemotherapeutic agent, and combinations thereof. In certain embodiments, the one or more bioactive factors is selected from the group consisting of Activin A and Activin B, Inhibin A, Inhibin B, TGF-α, TGF-β1, TGF-β2, TGF-β3, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, BMP-19, BMP-20, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, CDMP-1, CDMP-2, LMP-1, LMP-2, LMP-3, IGFBP1, IGFBP2, IGFBP3, IGFBP4, IGFBP5, IGFBP6, and kartogenin. The first layer can optionally further comprise one or more bioactive factors selected from the group consisting of TGFα, TGFβ, and kartogenin; and the second layer can optionally further comprise one or more bioactive factors selected BMP-2, BMP-3, BMP-4, BMP-5, BMP-6, BMP-7, BMP-8, and BMP-9. In certain embodiments, the first layer further comprises TGF-β1 and the second layer further comprises BMP-2.

As discussed herein, the tensile modulus and the compressive modulus of the multi-layered scaffold described herein can be altered by modifying the structure of the first and second tri-block poly(lactide-co-propylene glycol-co-lactide) dimethacrylate and the weight percentage of the functionalized hydroxyapatite nanoparticles. In certain embodiments, the multi-layered scaffold has a tensile modulus between 1-300 MPa and a compressive modulus between 10 and 500 MPa.

Advantageously, the topology of one or more exterior surfaces of the multi-layered scaffold can be modified to incorporate features to improve cell growth. The first layer and/or the second layer can optionally be molded during photopolymerization thereby fabricating a multi-layered scaffold comprising a cartilage phase layer and/or bone phase layer having an exterior surface comprising microfeatures selected from the group consisting of microgrooves, micropillars, microdots, microfibers, microcones, micropits, microspikes, and combinations thereof. The microfeatures can each range in size from 1-300 μm, 1-200 μm, 1-100 μm. In certain embodiments, an exterior surface of the cartilage phase layer comprises microgrooves and an exterior surface of the bone phase layer comprises micropillars, wherein the microgrooves range in width from 40-120 μm, 80-120 μm, 40-80 μm, and the micropillars have a diameter of 1-4 μm, 2-4 μm, or 2-3 μm and a height of 2-6 μm, 2-5 μm, or 3-5 μm.

Also provided is a method of fabricating the multi-layer scaffolded described herein, the method comprising: providing a first layer precursor; providing a second layer precursor; and photopolymerizing the first layer precursor and the second layer precursor thereby forming the multi-layer scaffolded, wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof. In certain embodiments, the method further comprises sequentially depositing the second layer precursor and the first layer precursor on a substrate.

In certain embodiments, the method comprises: depositing a second layer precursor on a substrate thereby forming the second layer; depositing a first layer precursor on a surface of the second layer thereby forming the first layer and the multi-layer scaffold precursor; and photopolymerizing the multi-layer scaffold precursor thereby forming the multi-layer scaffolded, wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator wherein the nanoparticles comprise hydroxyapatite, tricalcium phosphate, silicon dioxide, or bioglass.

The first layer precursor and/or the second layer precursor can be deposited neat or in solution.

The substrate can be a surface of an implantable medical device, glass, a polymer, a metal, an alloy, or a combination thereof.

The present disclosure also provides a method of repairing an OC defect in a subject in need thereof, the method comprising: depositing a second layer precursor at the OC defect site thereby forming a second layer; depositing a first layer precursor on a surface of the second layer thereby forming the first layer and the multi-layer scaffold precursor; and photopolymerizing the multi-layer scaffold precursor thereby forming the multi-layer scaffolded described herein, wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, hydroxyapatite covalently conjugated to the one or more hydroxyethyl methacrylate moieties via an optional linker, and the photoinitiator wherein the nanoparticles comprise hydroxyapatite, tricalcium phosphate, silicon dioxide, or bioglass.

The first layer precursor and/or the second layer precursor can be deposited neat or in solution.

The step of photopolymerization in the methods described herein can comprise irradiating the second layer and the first layer sequentially and/or simultaneously (i.e., photopolymerizing the multi-layer scaffold precursor) with electromagnetic radiation (e.g., ultraviolet light). In certain embodiments, the second layer is partially photopolymerized prior to deposition of the first layer before subjecting the partially photopolymerized second layer precursor coating and the first layer precursor coating to photopolymerization. In certain embodiments, the second layer and the first layer are first deposited and then the multi-layer scaffold precursor is photopolymerized.

The methods described herein can further comprise the step of shaping one or more of the first layer, the second layer, and the multi-layer scaffold precursor using a press or a negative mold prior to deposition.

Methods for fabricating molds for use in molding an exterior surface of the multi-layered scaffold are well known in the art. For example, a negative PDMS mold can be patterned by imprinting the surface of the PDMS mold with a mold comprising the desired topological features fabricated using a lithography method selected from the group consisting of electron-beam lithography, optical lithography, ultraviolet lithography, ion beam lithography, X-ray lithography, interference lithography, scanning probe lithography, charged particle lithography, or nanoimprint lithography.

The multi-layer scaffold precursor described herein can be used to repair an OC defect at or near (e.g., less than 5 cm, less than 4 cm, less than 3 cm, less than 2 cm, or less than 1 cm) the OC defect site. The OC defect can be the result of an injury, surgery, infection, malignancy, developmental malformation, and degenerative diseases such as osteoarthritis. In certain embodiments, the OC defect is the result of osteochondritis dissecans, articular collapse secondary to osteonecrosis or subchondral insufficiency fracture, osteochondral impaction fracture, or surgery.

The site of the OC defect can be at the femoral condyle, the humeral head, the talus, or the capitellum of the humerus.

In certain embodiments, deposition of the first layer precursor and the second layer precursor comprises injecting the first layer precursor and the second layer precursor at the site of the OC defect.

Synthesis and Characterization of Injectable PₘLₙDMA and PₘLₙDMA/MH

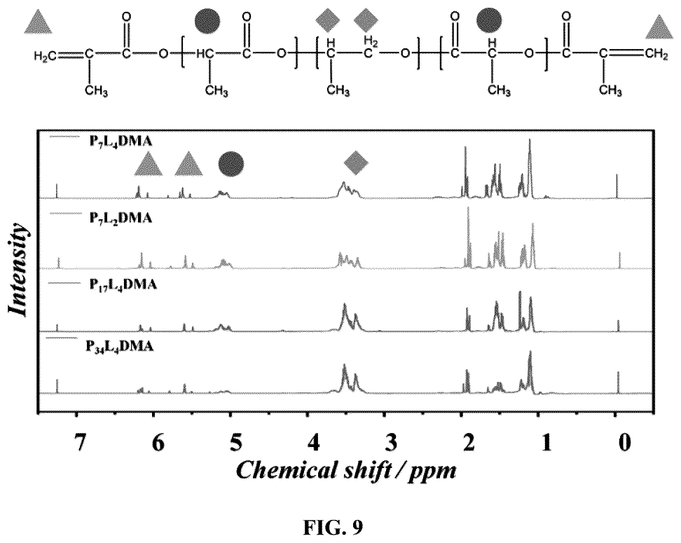

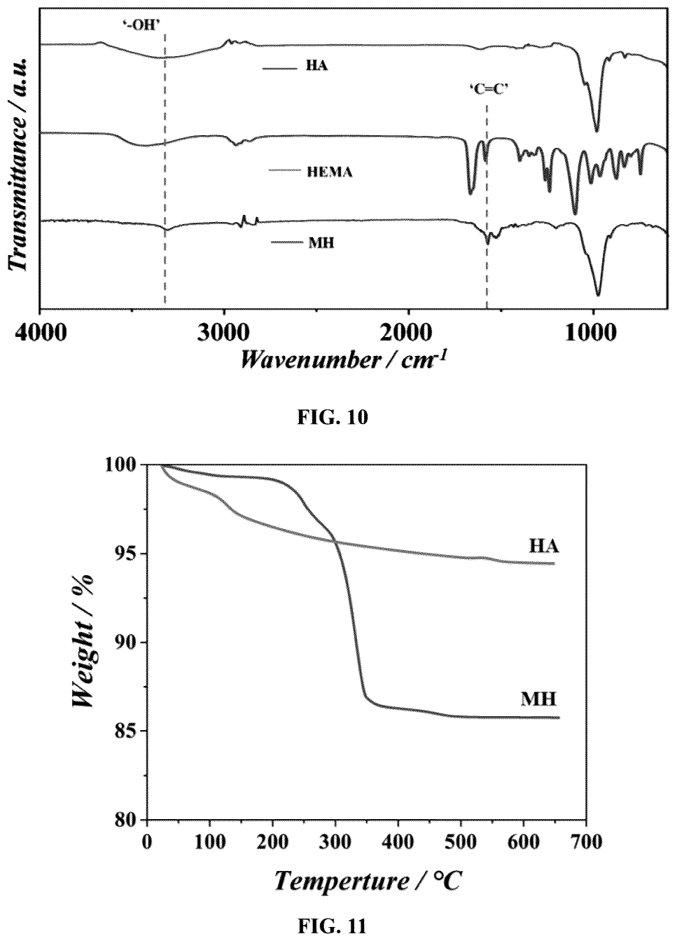

We first synthesized PₘLₙDMA with different compositions (m/n: 7/4, 7/2, 17/4, and 34/4) and MH to fabricate the injectable hydrophobic adhesives, followed by ¹H nuclear magnetic resonance (¹H NMR) and Fourier transform infrared spectroscopy (FTIR) analysis to validate the products. All PₘLₙDMA showed similar spectra with specific peaks around 1190 cm⁻¹, 1640 cm⁻¹, and 1747 cm⁻¹, corresponding to ‘C—O—C’, ‘C═C’, and ‘C═O’ stretches, respectively (FIG. 8). The ¹H NMR spectra showed peaks around 5.6 and 6.21 ppm, indicating the successful methacrylation of the PₘLₙDMA (FIG. 9). The synthesis efficiency of all compositions of PₙLₘDMA was calculated using ¹H NMR spectra with over 70% final efficiency (FIG. 30). In addition, FTIR analysis of the synthesized MH showed the 3400 cm⁻¹ and 1032 cm⁻¹ peaks that corresponds to the ‘—OH’ and ‘—PO₄³⁻’ groups; they remain unchanged compared to the naked hydroxyapatite (HA). The new peak around 1640 cm⁻¹ corresponding to the ‘C═C’ bond indicated the successful conjugation of hydroxyethyl methacrylate (HEMA) chain onto the surface of HA (FIG. 10). We then used the thermogravimetric analyzer (TGA) to quantify the amount of HEMA grafted onto the MH and we found nearly 7.4 wt. % HEMA was coupled to the MH (FIG. 11). The above results validated the successful synthesis of both PₘLₙDMA and MH for the subsequent studies.

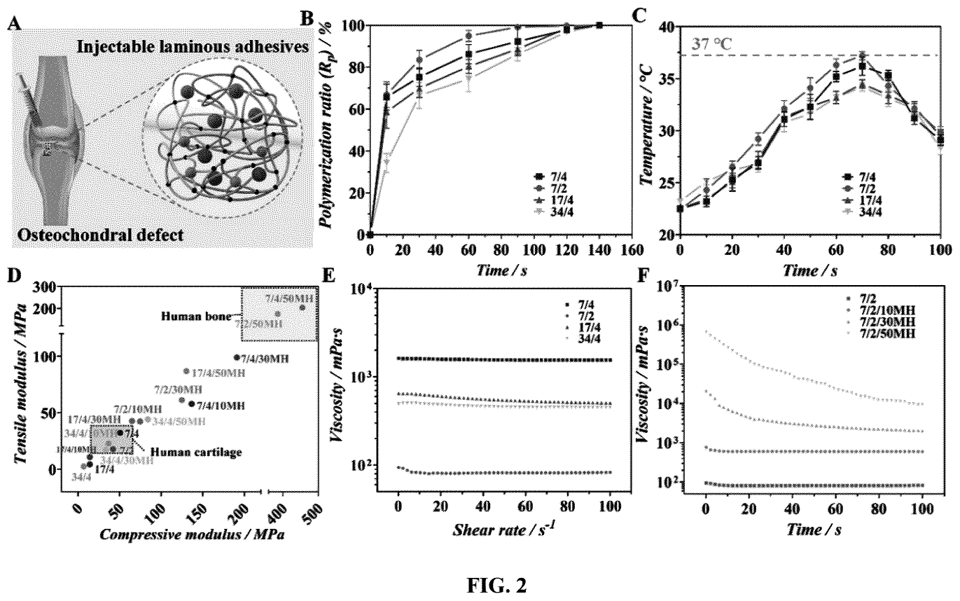

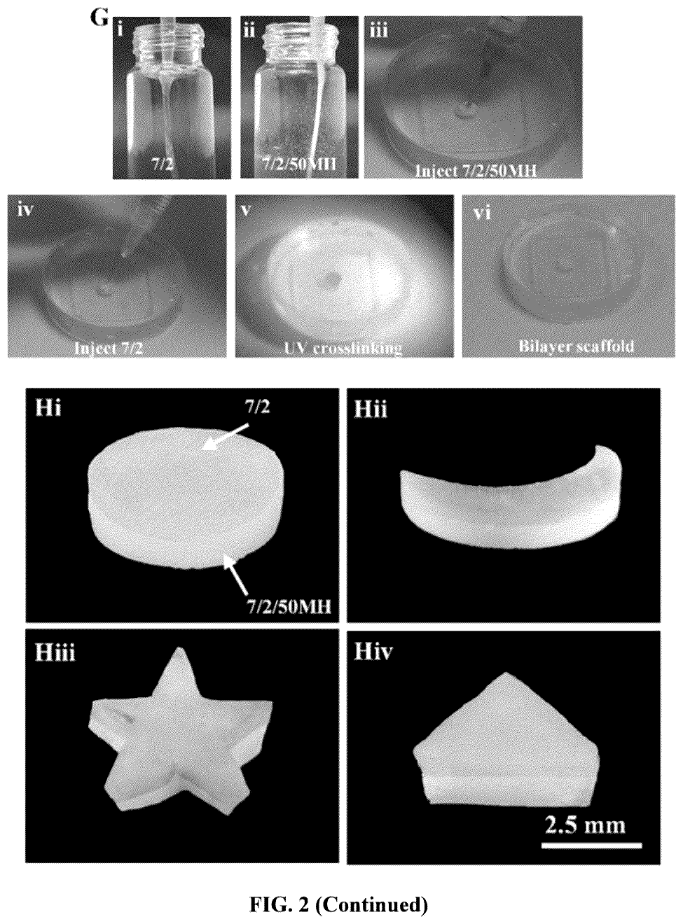

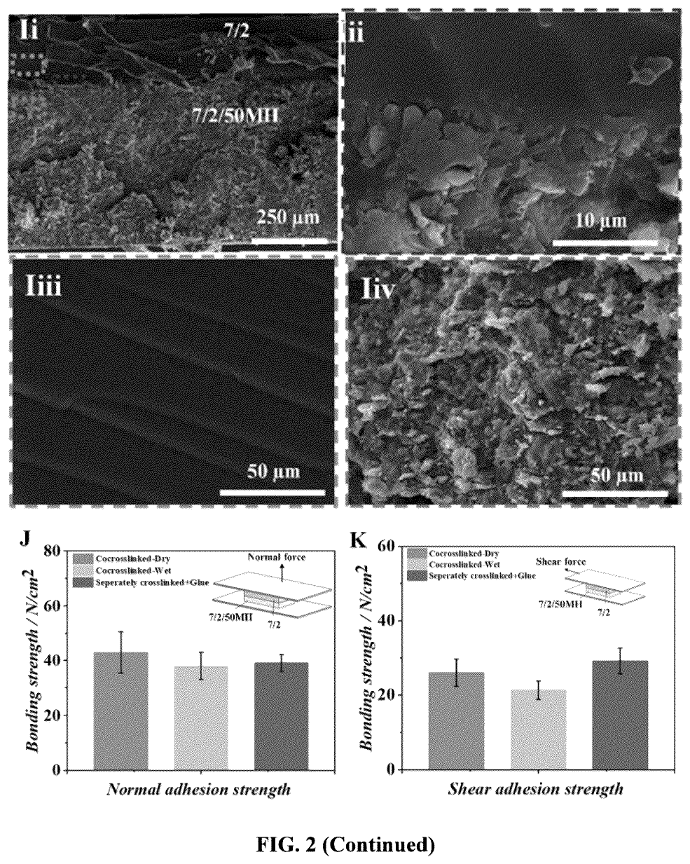

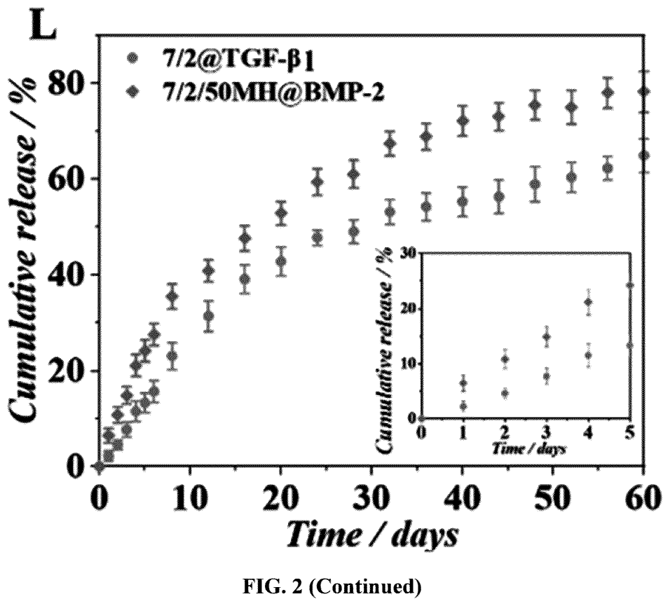

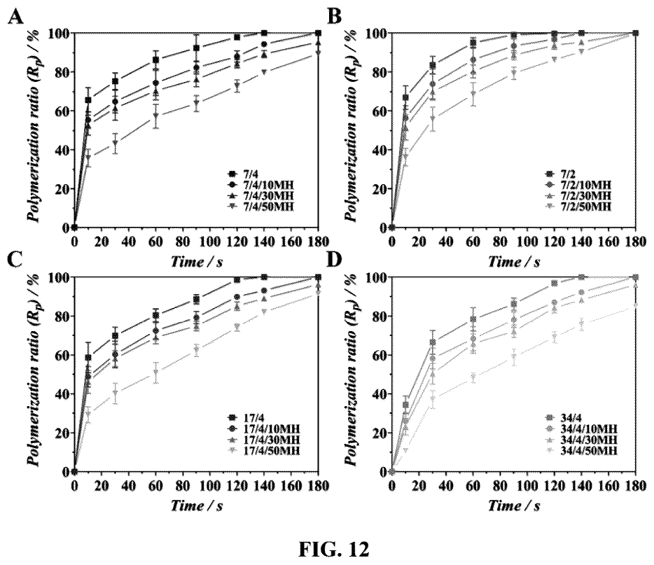

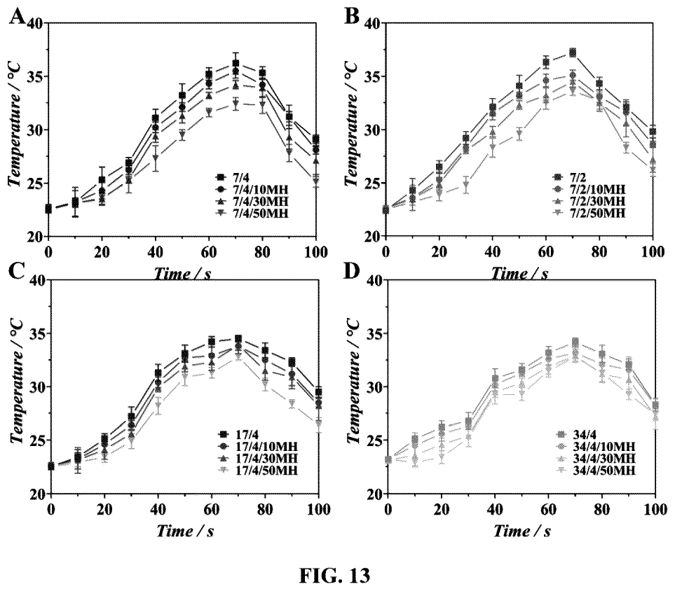

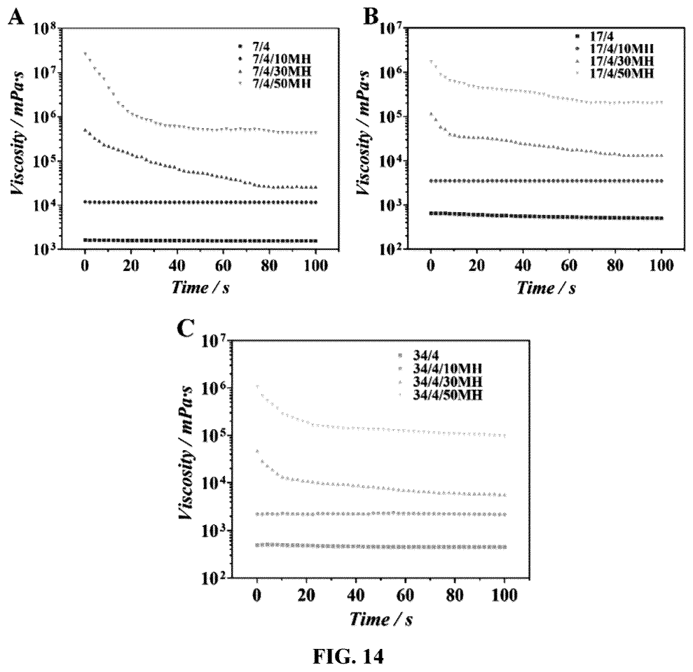

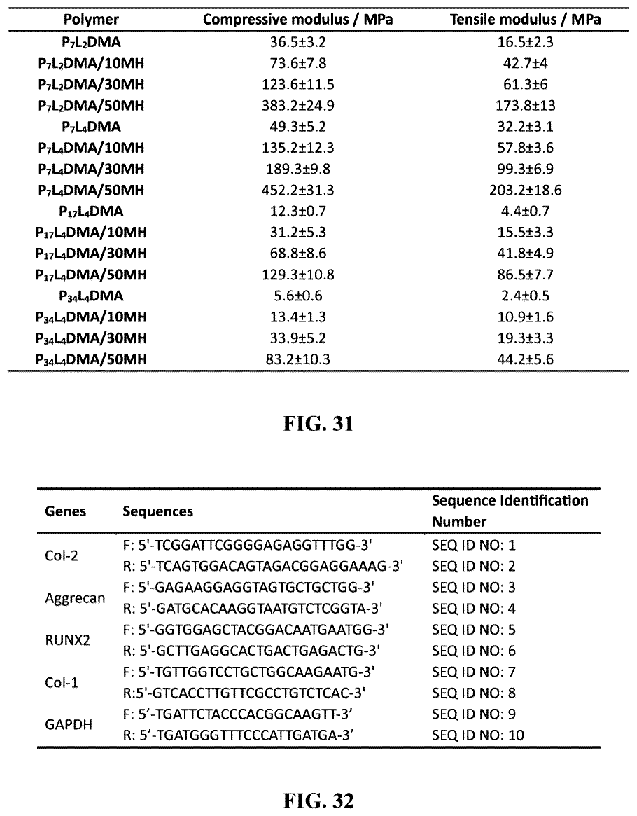

In our present study, we proposed to adopt the synthesized hyaline PₘLₙDMA and mineralized PₘLₙDMA/MH as cartilage and mineral bone phase respectively (FIG. 2A). Thus, we first evaluated the photopolymerization kinetics of the different adhesives under UV light (5 mW/cm²) using FTIR. All compositions of PₘLₙDMA adhesives underwent substantial polymerization within 20 s (FIG. 2B). P₇L₂DMA exhibited the fastest polymerization ratio (82.5 t 3.7% crosslinked for 30 s) due to the shorter polymer chain length and higher olefin concentration. Incorporating MH into the P₇L₂DMA (P₇L₂DMA/MH) slightly increased the time taken for photocrosslinking, attributed to its UV light scattering effect. Overall, all PₘLₙDMA and PₘLₙDMA/MH achieved photopolymerization within 180 s, demonstrating their capacity for rapid implementation (FIG. 12). Additionally, to confirm the controlled exothermic heat generation, we captured the temperature profile of the adhesives during the photopolymerization process using a thermal camera. The peak temperature for all adhesive formulations was lower than 37° C., suggesting their great potential for in vivo use and encapsulation of heat-labile therapeutic biomolecules (e.g., growth factors) (FIGS. 2C and 13). The mechanical properties of the adhesives play a vital role in stress-bearing and directing the commitment of MSCs to chondrocytes and osteoblasts. Thus, we tested the compressive and tensile modulus of different compositions of PₘLₙDMA and PₘLₙDMA/MH adhesives and plotted them in comparison with the natural cartilage and bone tissues (FIG. 2D and FIG. 31). We found the P₇L₂DMA/50MH and P₇LₙDMA/50MH presented similar compressive and tensile modulus to natural cancellous bone. Meanwhile, the mechanical properties of P₇L₂DMA, P₇L₄DMA, P₃₄L₄DMA/10MH, and P₃₄L₄DMA/30MH adhesives were similar to human cartilage^{[9, 14]}. To validate the injectability of the synthesized adhesives, we then analyzed the viscosity using a rheometer. We found that all compositions of PₘLₙDMA samples demonstrated Newtonian fluid characteristics with a constant viscosity regardless of shear rate (FIG. 2E). The P₇L₂DMA group presented the lowest viscosity of 100 mPa·s, while the P₇L₄DMA exhibited the highest viscosity of 1,000 mPa·s. The incorporation of MH into PₘLₙDMA could increase the viscosity of the composites (FIG. 2F). The P₇L₄DMA/50MH demonstrated over 106 mPa·s viscosity, rendering them a relatively poor candidate as an injectable adhesive for osteochondral repair (FIG. 14). Overall, the P₇L₂DMA and P₇L₂DMA/50MH adhesives demonstrated excellent injectability, rapid photocrosslinking with low heat generation, and matching mechanical properties to natural cartilage and cancellous bone. Therefore, we selected the P₇L₂DMA and P₇L₂DMA/50MH as the cartilage and cancellous bone phases, respectively, for the subsequent experiments.

### Fabrication and Characterization of Underwater Crosslinking Bi-Layered Adhesives

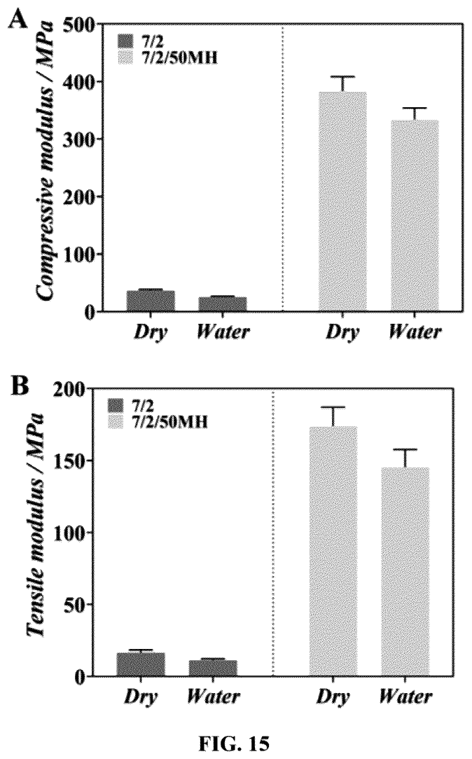

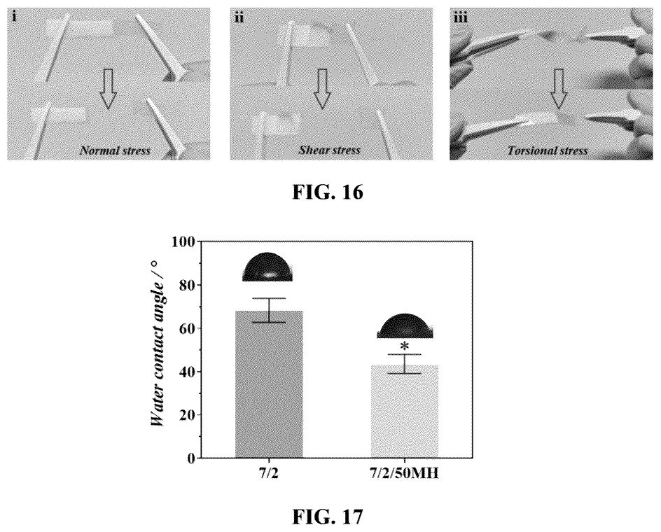

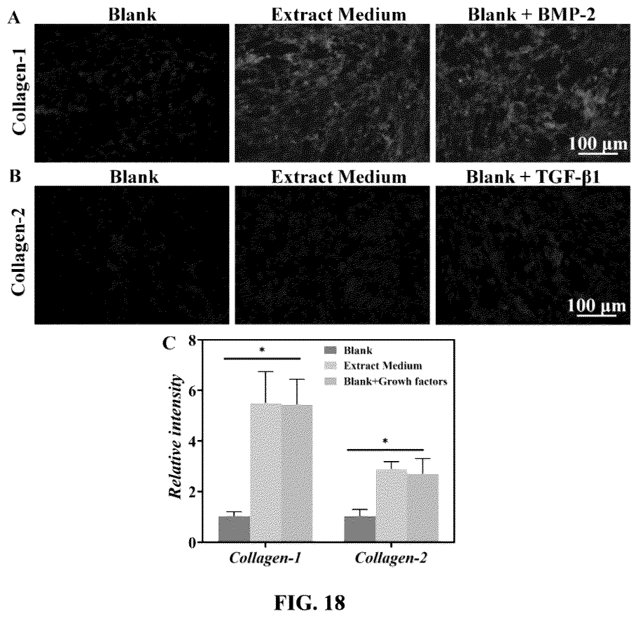

Clinically, the OC repairing process may expose the adhesives to fluid (e.g., irrigation and synovial fluid). Therefore, it is essential for the adhesives to be confined during injection without dispersal.^{[15]} Thus, we tested the under-water injectability of P₇L₂DMA and P₇L₂DMA/50MH by directly injecting them into water. Both adhesives were nicely confined within the injection site, indicating their capability for underwater applications (FIGS. 2Gi and 2Gii). We then characterized the subaqueous implementation performance of our adhesives in subaqueous condition. After sequential application of P₇L₂DMA/50MH and P₇L₂DMA and photocrosslinking, we found the adhesives could form the bi-layered structure subaqueously without significant difference from the dry condition (FIGS. 2Giii-Gvi). Moreover, the ability of the liquid adhesives to conform to the irregular structures of the defect site is crucial for its clinical application. To assess the moldability of our adhesives, we prepared different molds (e.g., moon, star, and triangle) to simulate the irregular geometry of the defect site and applied our adhesives subaqueously. We found the adhesives demonstrated excellent fluidity and could conform to different irregular molds and form bi-layered structures (FIG. 2Hi to Hiv). Furthermore, no significant differences in compressive or tensile modulus were found between the samples photocrosslinked under dry and subaqueous conditions, indicating that underwater crosslinking would not compromise the mechanical properties of the adhesives (FIG. 15). Additionally, the interfacial bonding is also of high interest as joint motility is a complicated process that calls for a robust bonding interface to maintain the stability and integrity of the bi-layered structure. Thus, we evaluated the interfaces of the bi-layered scaffolds using scanning electron microscopy (SEM) (FIG. 2I). The P₇L₂DMA layer presented hyaline-like smooth morphology (FIG. 2Iiii) while the PₘLₙDMA/MH layer appears mineralized morphology (FIG. 2Iiv), resembling the natural OC complex. The interface between the two adhesive layers appeared tight and seamless (FIG. 2Ii to ii). Quantitatively, we performed lap shear test to evaluate the interfacial bonding strength of the scaffolds. The P₇L₂DMA and P₇L₂DMA/50MH formed strong interfacial bonding, withstanding 43.1±7.6 N/cm² of normal force and 26.2±3.7 N/cm² of shearing force (FIGS. 2J and K). Under extreme axial, lateral and rotational forces, all bi-layered scaffolds broke at the mechanically weaker P₇L₂DMA layer, leaving the stronger P₇L₂DMA/50MH layer and the bonding interface intact, indicating the bonding interface could bear the different force from the joint movement after implantation (FIG. 16). Given the low exothermic heat of our adhesives, we proceed to evaluate their capacity to carry heat-labile bioactive molecules. We selected to incorporate transforming growth factor-beta 1 (TGF-β1) and bone morphogenetic protein-2 (BMP-2) in P₇L₂DMA and P₇L₂DMA/MH adhesives respectively due to their chondrogenic and osteogenic potential, forming 7/2@TGF-β1 (TGF-β1 in P₇L₂DMA) and 7/2/50MH@BMP-2 (BMP-2 in P₇L₂DMA/50MH) adhesives.^{[16]} We then incubated the two adhesives in phosphate-buffered saline (PBS) and measured the cumulative release of the loaded growth factors using ELISA kit. Both growth factors displayed a slow and long-term release over 60 days, attributed to the hydrophobic nature of the adhesives (FIGS. 2L and 17). Next, we validated the bioactivity of the released TGF-β1 and BMP-2 with collagen-1 (osteogenic marker) and collagen-2 (chondrogenic marker) immunofluorescence staining of rat bone marrow stem cells (rBMSCs)^{[17]}. We found the rBMSCs cultured using the extracted medium possessed comparable chondrogenesis or osteogenesis performance to medium with fresh growth factors, indicting the preserved bioactivity of growth factors (FIG. 18).

The above results confirmed the feasibility of our adhesive for the preparation of bi-layered OC scaffolds with excellent subaqueous implementation ability, strong interfacial bonding and long-term growth factor release capacity.

### Biocompatibility, Chondrogenesis, and Osteogenesis of Bi-Layered Adhesives

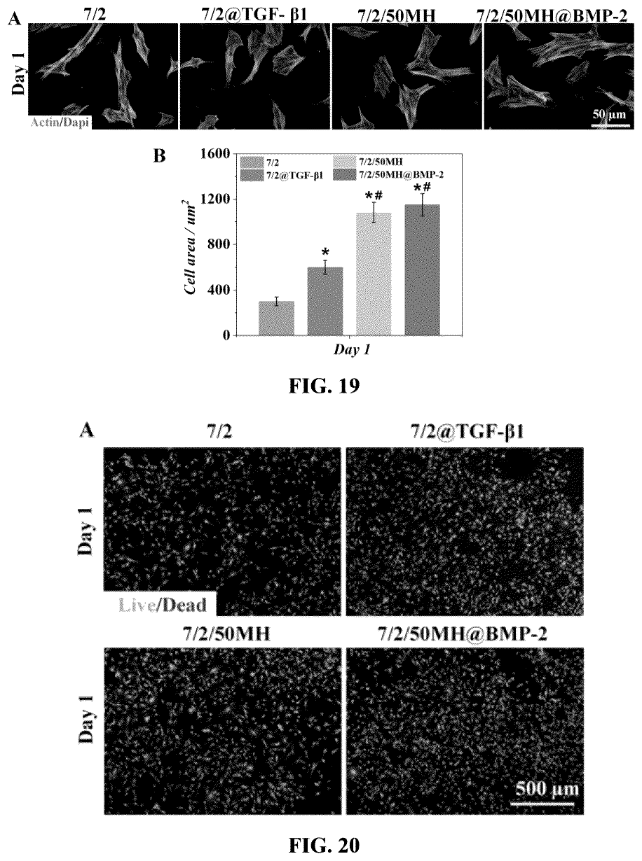

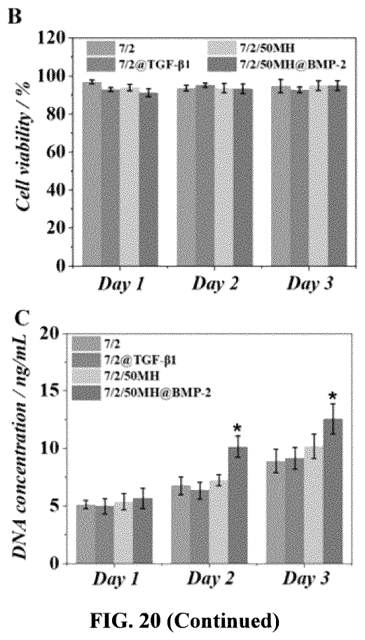

After successful preparation of the bi-layered adhesives, we then evaluated their biocompatibility by direct seeding the rBMSCs onto the adhesives. In addition to the 7/2@TGF-β1 and 7/2/50MH@BMP-2, we also used the P₇L₂DMA (denoted as 7/2) and P₇L₂DMA/50MH (denoted as 7/2/50MH) as the controls. The 7/2@ TGF-β1 adhesive was mixed with 3 ug TGF-β1 in 1 g of 7/2 polymers, which could achieve sufficient release concentration of TGF-β1 for BMSCs' chondrogenic differentiation.^{[18]} The 7/2/50MH@BMP-2 adhesive was mixed with 30 μg BMP-2 in 1 g 7/2/50MH nanocomposites, which could achieve suitable release concentration of BMP-2 for BMSCs' osteogenic differentiation according to our previous research.^{[7]} F-actin staining revealed that all cells adhered well to the adhesives on day 1. The MH incorporated groups (7/2/50MH and 7/2/50MH@BMP-2) showed 3.6-fold and 3.8-fold increased cell spreading area compared to the 7/2 group (FIG. 19). It has been demonstrated that MSCs displayed larger spreading area on stiff substrate than that on the soft substrate.^{[19]} Moreover, all groups presented over 90% viability and stable proliferation after 3 days of incubation (FIG. 20 A-C). Consistent with previous studies, BMP-2 incorporated adhesives (7/2/50MH@BMP-2) showed enhanced cell proliferation.^{[20]} These results indicated the excellent biocompatibility of the adhesives for OC defect implantation.

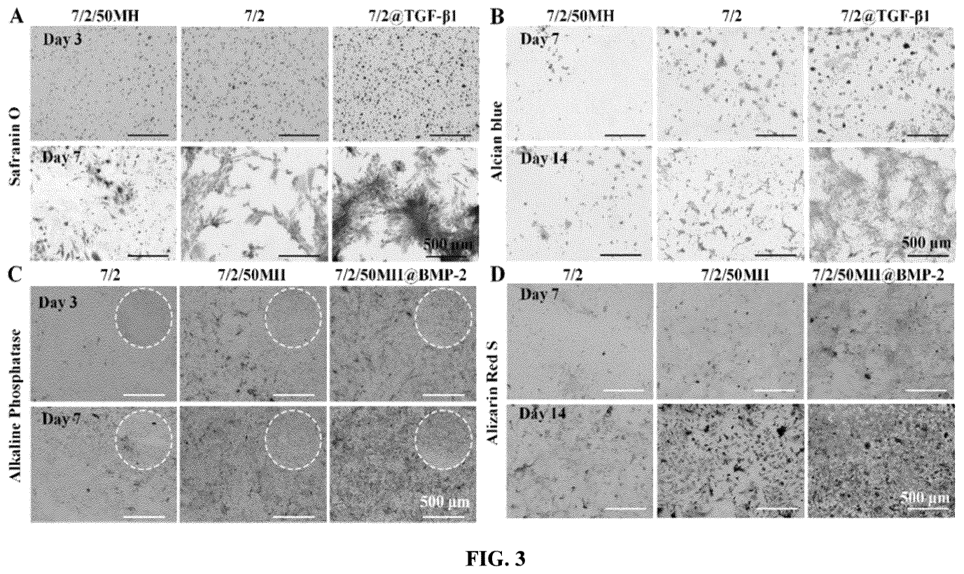

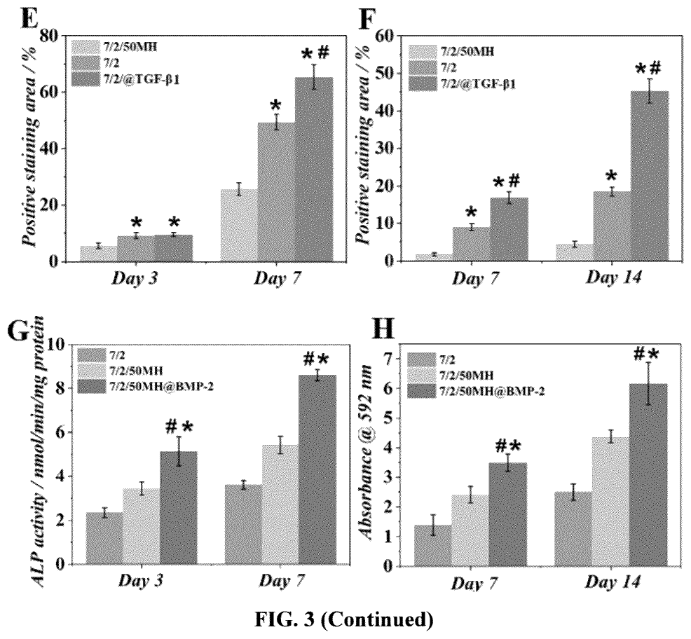

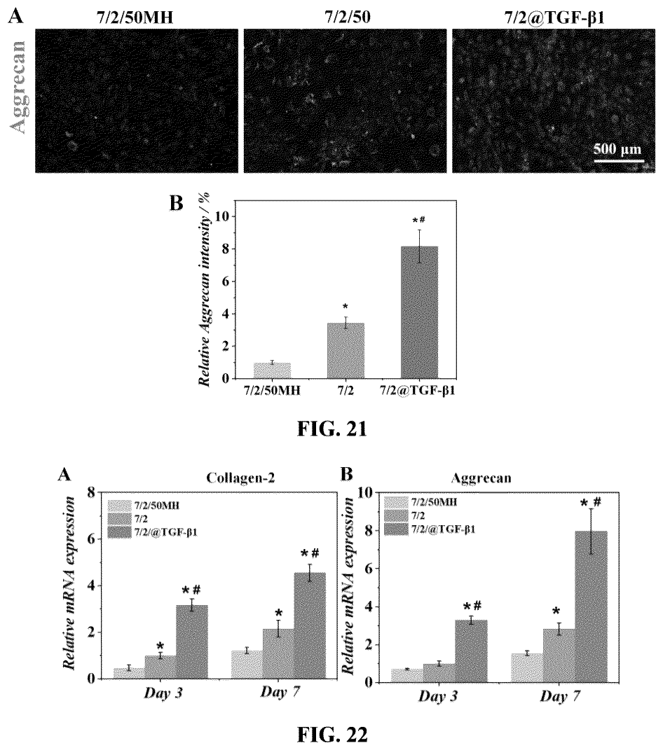

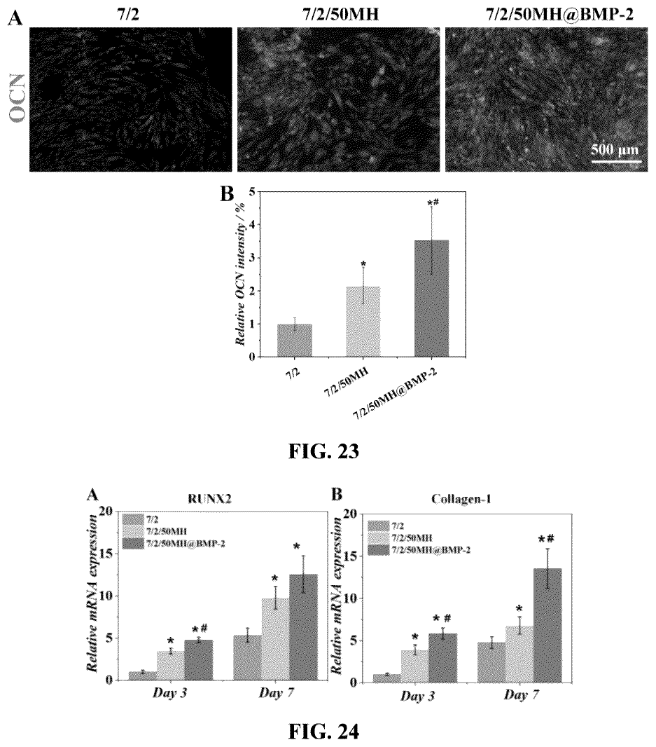

Next, we evaluated the chondrogenesis of the cartilage phase by culturing rBMSCs onto the 7/2, 7/2/50MH and 7/2@TGF-β1 adhesive discs with chondrogenic medium. Safranin O staining on day 3 and 7 post-incubation showed a 1.9- and 2.5-fold increase in glycosaminoglycans in soft 7/2 and 7/2@TGF-β1 groups compared to the stiff 7/2/50MH substrate (FIGS. 3A and E). Alcian blue staining on day 7 and 14 post-incubation showed significantly higher proteoglycan production and chondrogenesis in 7/2@TGF-β1 group compared to other groups (FIGS. 3B and F). Immunofluorescence staining of aggrecan showed stimulated aggrecan synthesis in the 7/2 group when compared to the 7/2/50MH group, which was further enhanced in the 7/2@TGF-β1 group (FIG. 21). The mRNA expression of Collagen-2 and aggrecan also showed consistent results that 7/2@TGF-β1 group enhanced the chondrogenesis gene expression (FIG. 22). Similarly, we evaluated the osteogenesis of the subchondral bone phase by culturing rBMSCs onto the 7/2, 7/2/50MH, and 7/2/50MH@BMP-2 adhesive discs with osteogenic medium. Alkaline phosphatase (ALP) staining performed on day 3 and 7 showed enhanced osteogenesis in the 7/2/50MH group, and was further promoted in the 7/2/50MH@BMP-2 group (FIG. 3C). The ALP activity of 7/2/50MH@BMP-2 group was 1.5- and 2.4-fold higher than the 7/2 and 7/2/50MH groups on day 7, respectively (FIG. 3G). Consistently, Alizarin Red S (ARS) staining on day 7 and 14 showed significant promotion of late calcium deposition in MH and BMP-2 incorporated substrates (FIGS. 3D and H). Promoted osteogenesis was also observed in the immunofluorescence staining of key osteogenic marker of osteocalcin (OCN). The 7/2/50MH@BMP-2 group showed significantly heightened OCN expression compared to other groups (FIG. 23). The mRNA expression of RUNX2 and collagen-1 also showed consistent results that 7/2/50MH@BMP-2 group enhanced the osteogenesis gene expression (FIG. 24). Altogether, we confirmed our bi-layered OC adhesives endowed with corresponding biophysical and biochemical cues exhibited synergistical chondrogenic and osteogenic promotion for the OC regeneration.

Bioinformatic Analysis of rBMSCs on Bi-Layered Adhesives

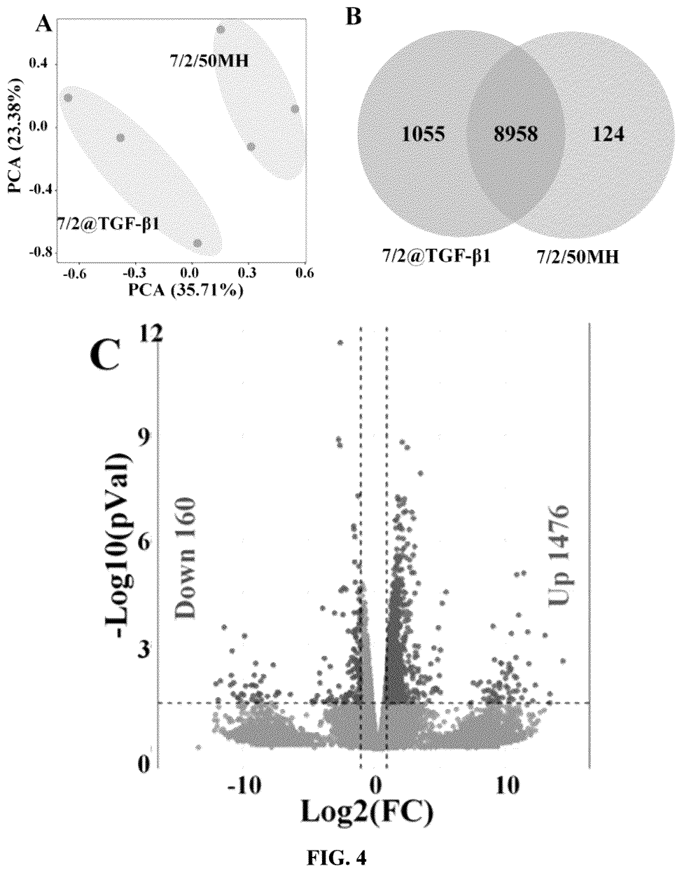

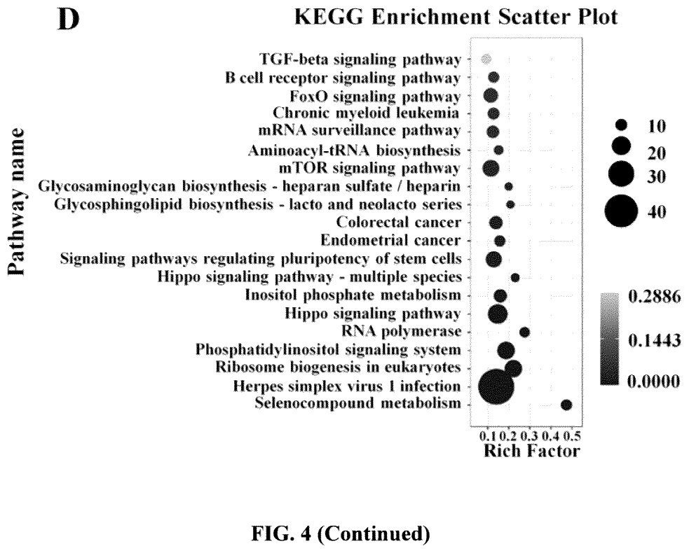

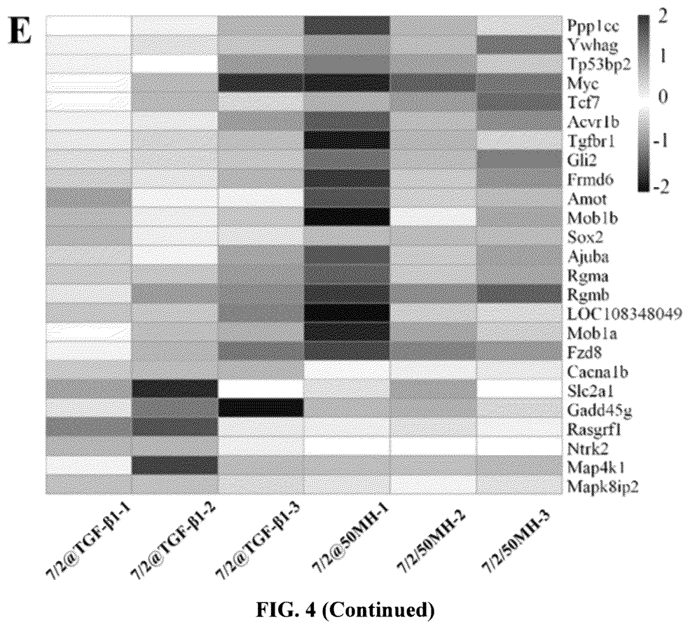

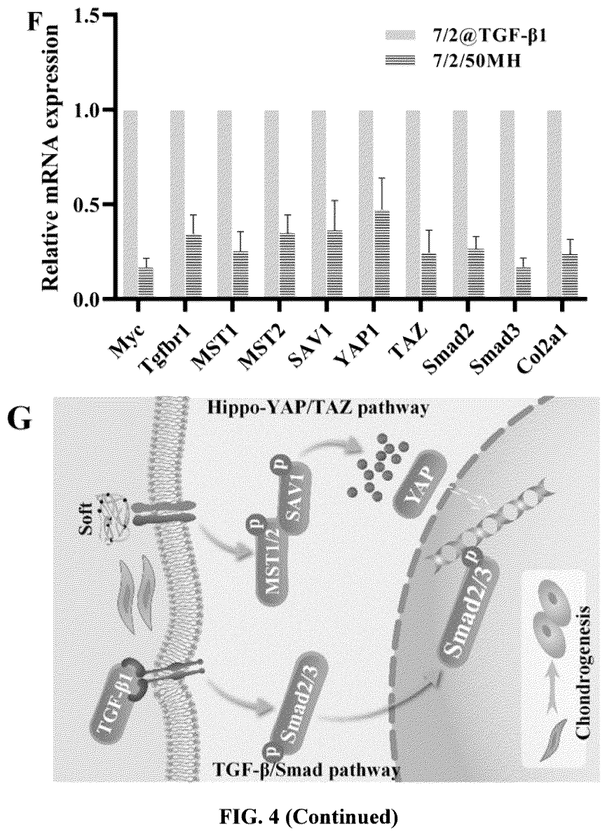

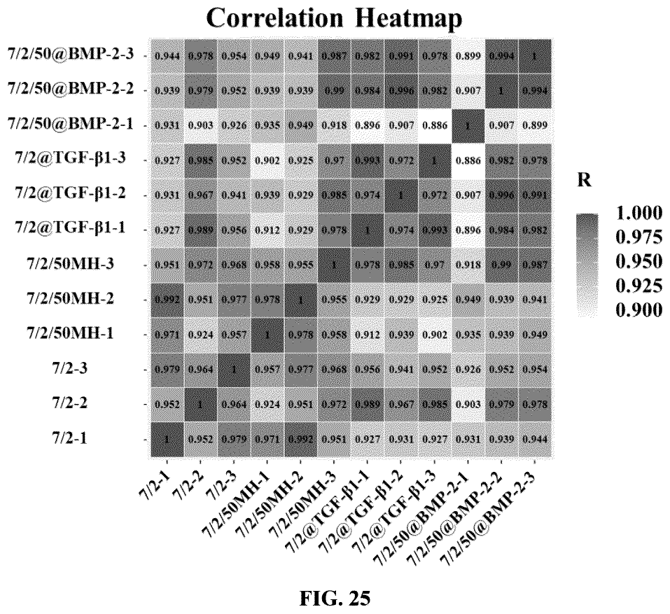

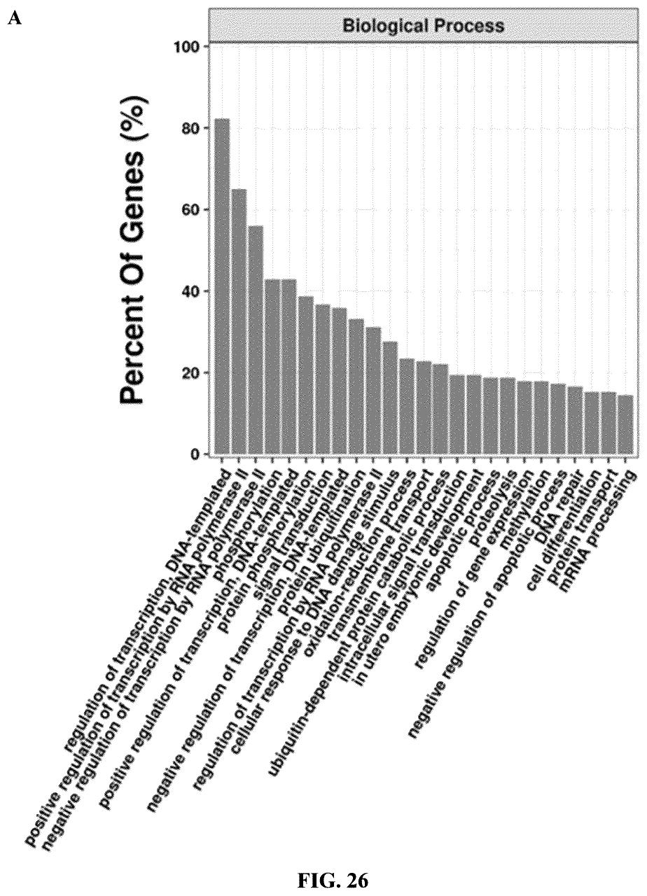

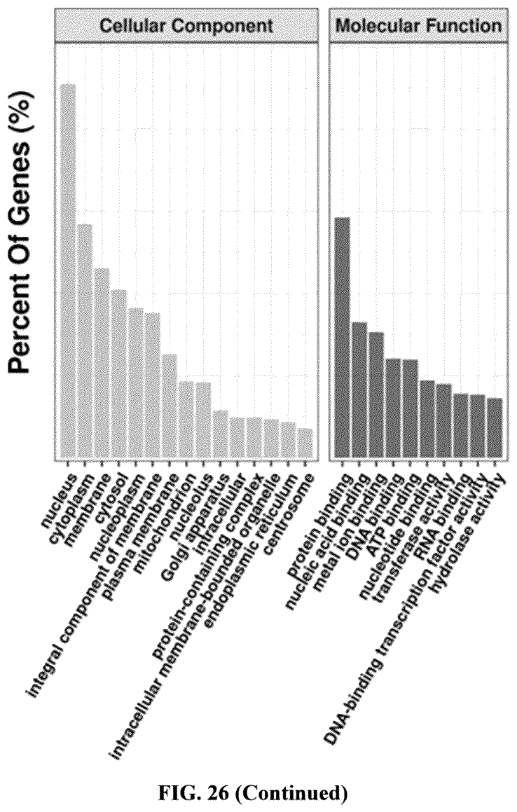

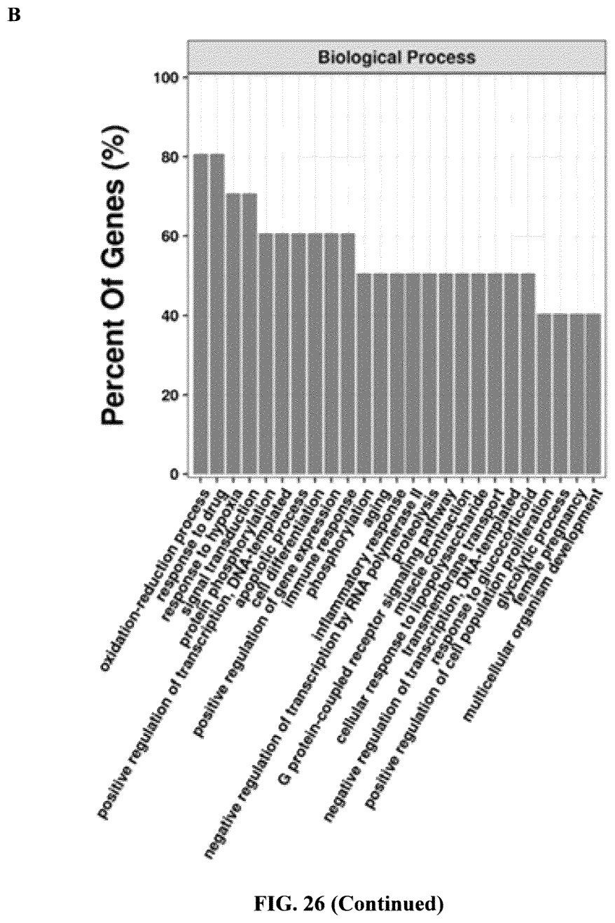

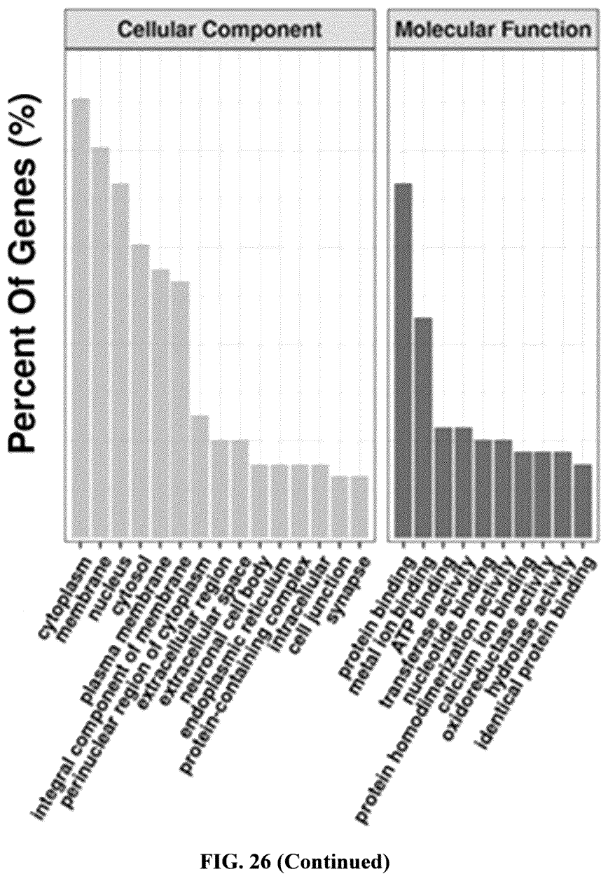

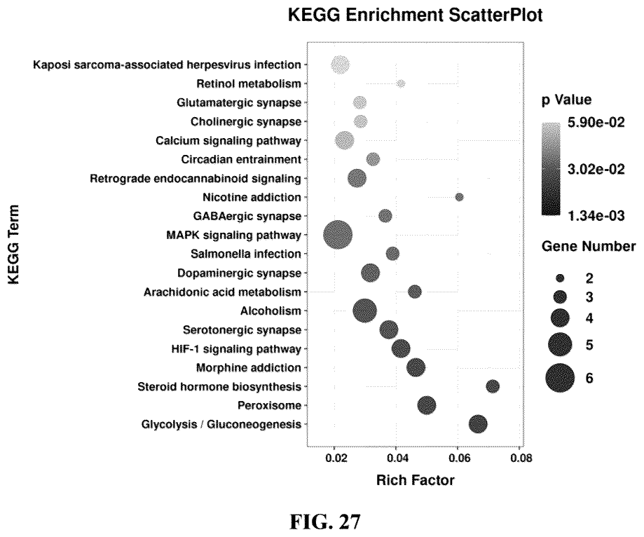

To elucidate the underlying mechanism of the above mentioned synergistically osteogenic and chondrogenic differentiation enhancement of rBMSCs, we performed transcriptomic analyses of rBMSCs cultured on two sets of adhesives. The first set comprised of 7/2@TGF-β1 and 7/2/50MH adhesives to examine the effect of soft ECM and TGF-β1 on chondrogenesis. The Pearson correction and principal component analysis (PCA) shown good specimen's stability between two groups (FIG. 25 and FIG. 4A). Then, the differential gene expression (DGE) was evaluated. A wide DGEs was observed between the 7/2@TGF-β1 and 7/2/50MH adhesives (1055 in 7/2@TGF-β1 and 124 in 7/2/50MH) (FIG. 4B). The volcano plots additionally showed 1476 up-regulated and 160 down-regulated genes (FIG. 4C). The above results indicated the presence of gene expression changes in response to the biophysical (e.g., stiffness of adhesives) and biochemical cues (e.g., TGF-β1). We then took the DEGs for further Gene Ontology (GO) database evaluation which included biological process (BP), molecular function (MF), and cellular component (CC). The significantly enriched upregulated GO terms including protein phosphorylation, cell differentiation and protein transport indicated that 7/2@TGF-β1 adhesives could modulate the rBMSCs' differentiation via protein phosphorylation (FIG. 26). We further conducted Kyoto Encyclopedia of Genes and Genomes (KEGG) pathway analysis on the DEGs, revealing significant upregulation of the chondrogenesis related pathways including TGF-β signaling pathway and Hippo signaling pathway, and downregulation of the HIF-1 and MAPK signaling pathways (FIGS. 4D and 27). In addition, the heatmap also revealed a series of genes was significantly up-regulated (e.g., Myc, Tgfbr1, Acvr1b) or down-regulated (e.g., Slc2a1, Map4k1, Mapk8ip2) in these relevant signaling pathways (FIG. 4E). To validate these gene expression data, we followed with quantitative real-time polymerase chain reaction (qRT-PCR) analysis and observed consistent regulation of targeted RNA-seq expression in related signal pathways. The expression of MST1/2, SAV, YAP/TAZ and Smad2/3 genes in 7/2@TGF-β1 were significantly higher than that of the 7/2/50MH group (FIG. 4F). The above transcriptomic results collectively showed significant upregulation of Hippo and TGF-β pathway, which have been widely reported to play a role in stem cell chondrogenic differentiation. Therefore, we hypothesized that the 7/2@TGF-β1 adhesives promoted chondrogenesis through the crosstalk between the TGF-β1 pathway and the mechanotransduction Hippo-YAP/TAZ pathway. In detail, the TGF-β1 in 7/2@TGF-β1 could prime the TGF-β pathway in favoring chondrocyte differentiation by promoting the downstream phosphorylation of Smad2/3, which could mediate chondrocyte differentiation^{[21]}. However, the phosphorylated Smad2/3 could be inhibited by YAP/TAZ in nucleus with compromised efficacy.^{[22]} Fortunately, the soft ECM in 7/2@TGF-β1 stimulated the integrin-dependent Hippo signaling which could cause the phosphorylation and degradation of YAP/TAZ in the cytoplasm. The degradation of YAP/TAZ could relieve the inhibition of Smad2/3 to enhance the chondrocyte differentiation.^{[23]}

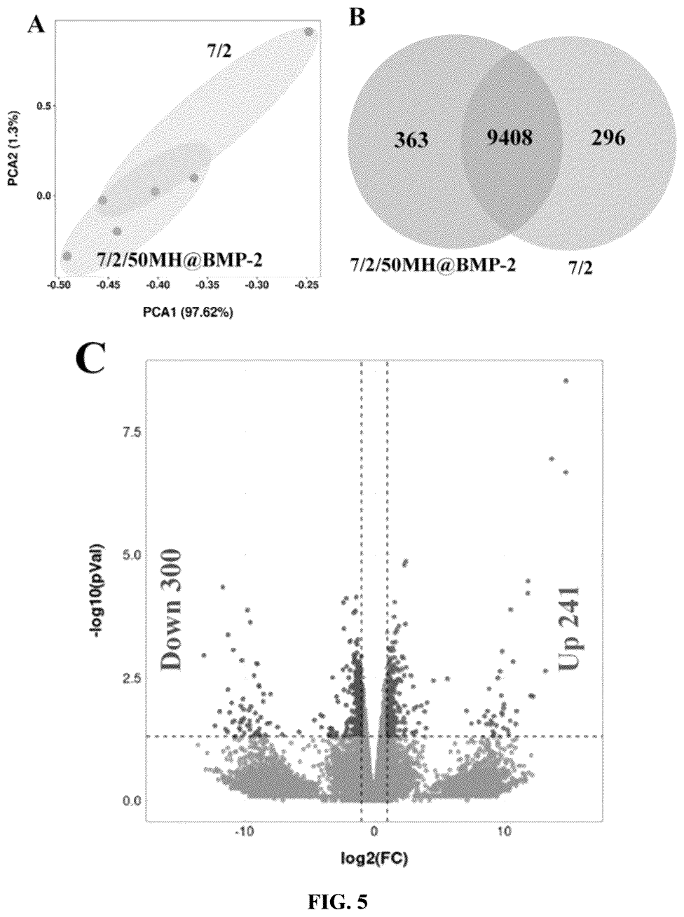

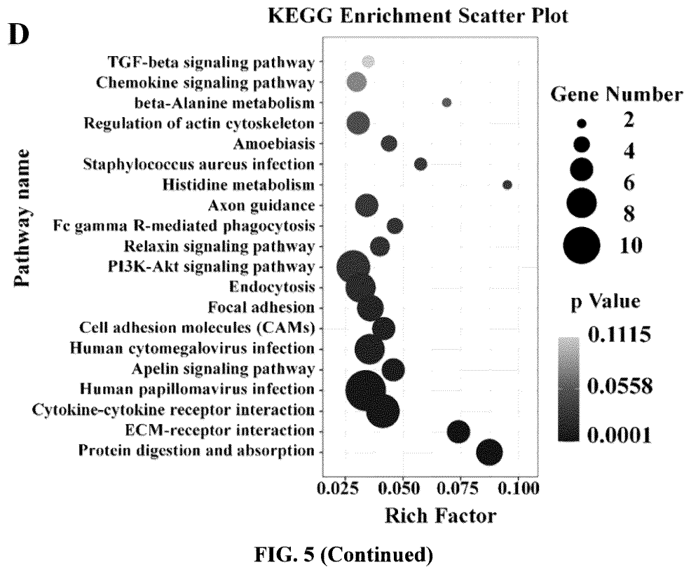

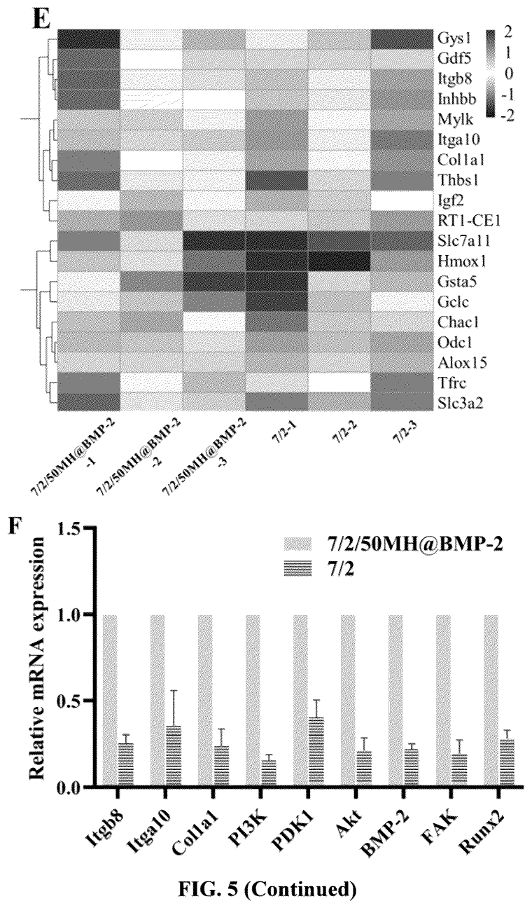

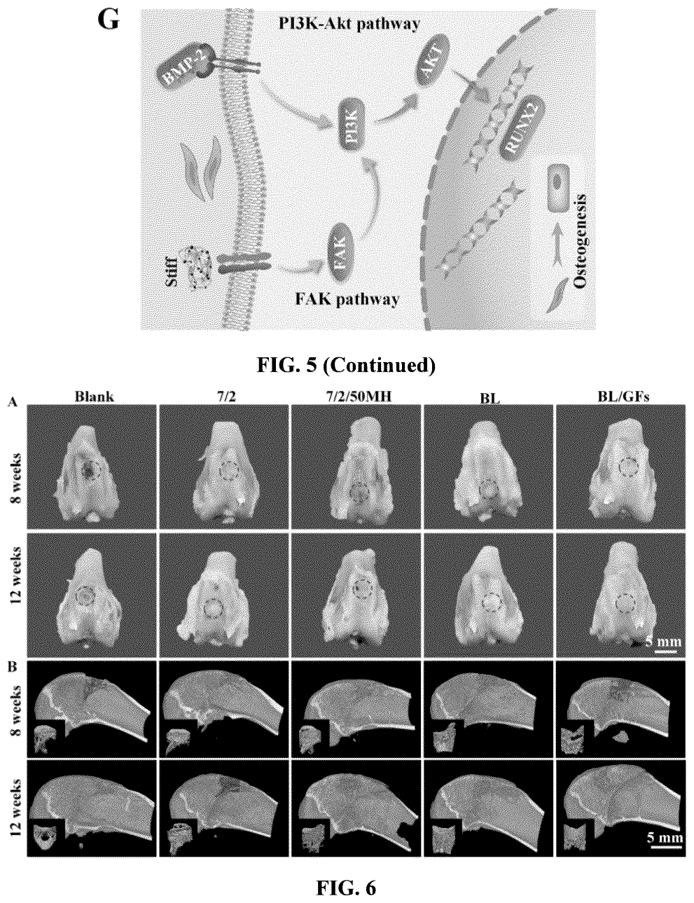

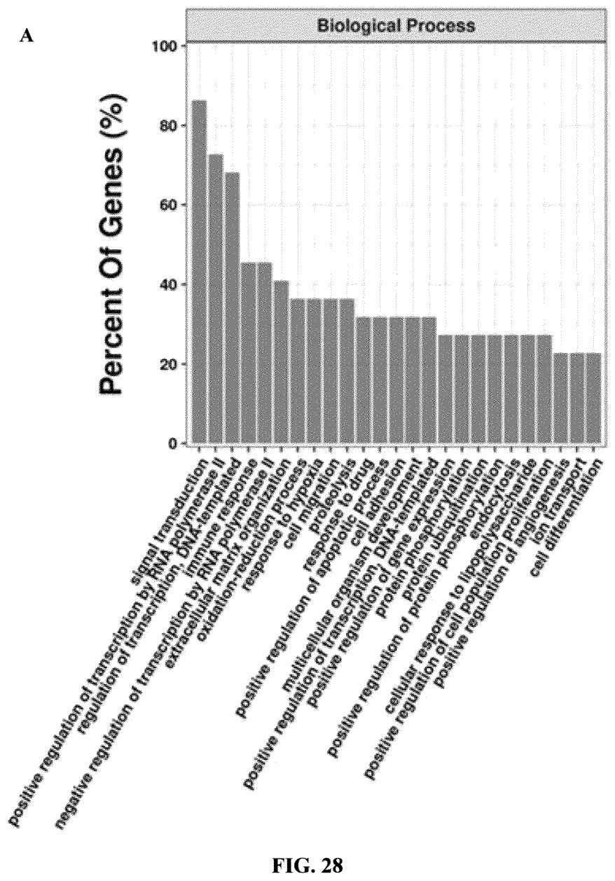

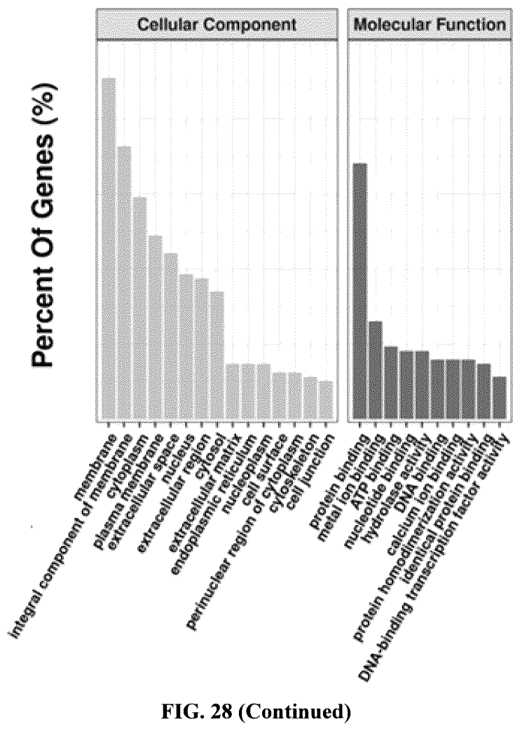

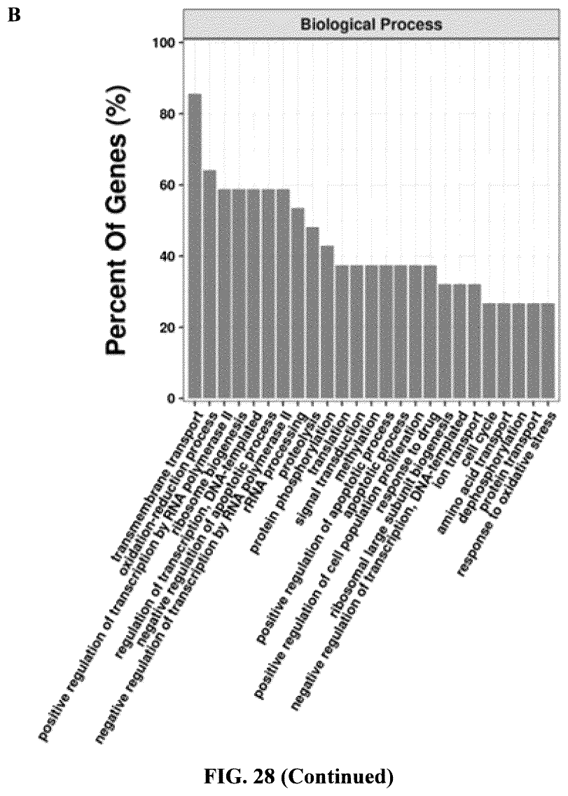

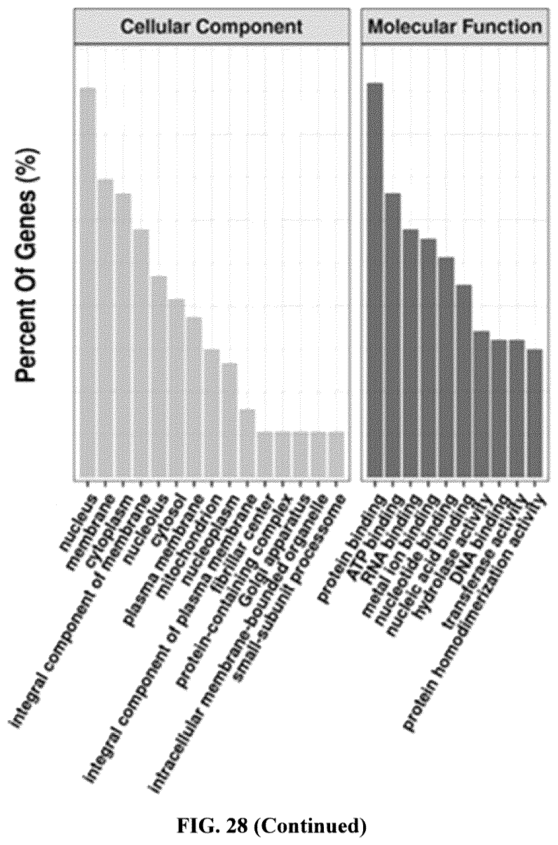

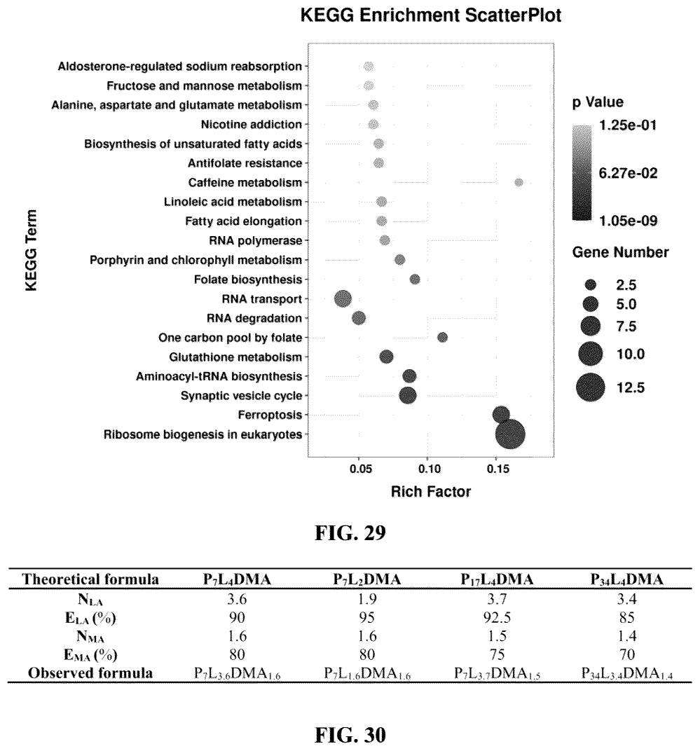

The second set comprised of rBMSCs cultured on 7/2/50MH@BMP-2 and 7/2 adhesives to examine the effect of stiff ECM and BMP-2 on osteogenesis. Similarly, Pearson correction and principal component analysis (PCA) showed good specimen's stability between two groups (FIG. 25 and FIG. 5A). Meanwhile, a wide DGEs was also observed between the two groups (363 in 7/2/50MH@BMP-2 and 296 in 7/2) (FIG. 5B). The volcano plots showed 241 upregulated and 300 down-regulated genes (FIG. 5C). Further GO database evaluation of significantly enriched GO terms demonstrated key osteogenesis related upregulation in ECM protein binding, regulation of calcium ion transport and regulation of cell adhesion (FIG. 28). KEGG pathway analysis of the DEGs between the 7/2/50MH@BMP-2 and 7/2 groups revealed significant up-regulation of the PI3K-Akt signaling pathway, focal adhesion pathway, as well as the ECM-receptor interaction pathway and down-regulation of the Glutathione metabolism and Ferroptosis signal pathways (FIGS. 5D and 29). Heatmap of DEGs also showed a series of genes were significantly upregulated (e.g., Gdf5, Itgb8, Itga10, Col1a1) or downregulated (e.g., Slc7a11, Gsta5, Slc3a2) (FIG. 5E). To validate these signaling pathways, we further performed qRT-PCR to validate the targeted gene expression in related signal pathways. The expression of FAK, PI3K, Akt and RUNX2 genes in 7/2/50MH@BMP-2 were significantly higher than that of the 7/2 group (FIG. 5F). The above transcriptomic results collectively demonstrated significant upregulation of the PI3K-Akt, FAK, and ECM-receptor interaction pathways, which have been widely reported to drive osteo-precursor development of MSCs. Therefore, we hypothesized that the 7/2/50MH@BMP-2 adhesives promoted the osteogenesis through the crosstalk between the BMP-2 pathway and the mechanotransduction FAK pathway with the co-activation of the PI3K-Akt. In detail, the BMP-2 in 7/2/50MH@BMP-2 could firstly upregulate its downstream PI3K-Akt. The PI3K-Akt has been widely reported to drive the osteogenesis process by facilitating the transcriptional activities of RUNX2 to stimulate ALP activity.^{[24]} Simultaneously, the stiff 7/2/50MH@BMP-2 adhesives could also activate the integrin mediated FAK pathways, which could further upregulate the downstream PI3K-Akt to further enhance the osteoblast differentiation of the rBMSCs.^{[25]} Altogether, the above results illustrated the ability of our adhesive to synergistically modulate the crosstalk between mechanotransduction and growth factor signaling pathways, providing an insight into their chondrogenesis and osteogenesis promotion.

### In Vivo Osteochondral (OC) Regeneration Efficacy of the Bi-Layered Adhesives

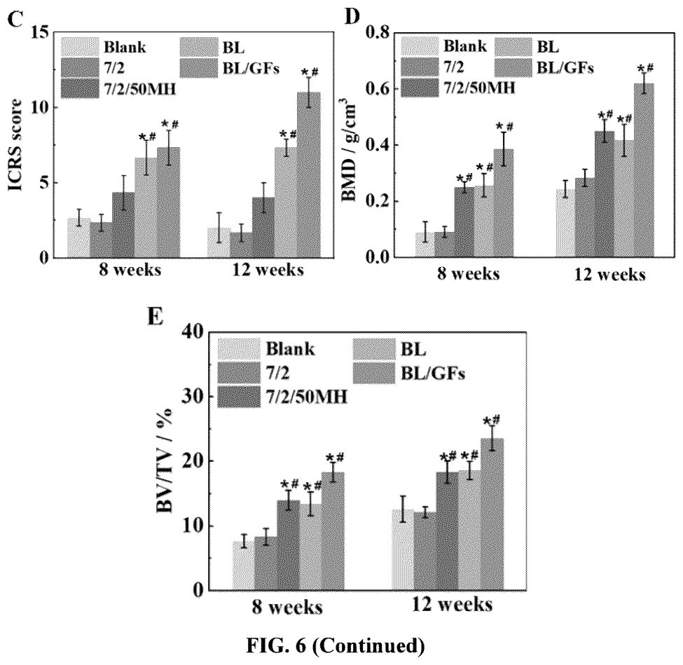

While previous characterizations demonstrated the bi-layered adhesives with promising in vitro OC regeneration efficacy, its long-term clinical relevance under physiological conditions remains unexplored. We thus next characterized the in vivo therapeutic performance of the bi-layered adhesives using a rabbit OC defect model. We divided the rabbits into the following five groups: blank, 7/2, 7/2/50MH, BL (bi-layered adhesives), and BL/GFs group (bi-layered adhesives loaded with TGF-β1 and BMP-2 in the cartilage and bone phase, respectively). For the blank, 7/2, and 7/2/50MH groups, we injected the corresponding composite into the defect site. For the BL and BL/GLs groups, we first layered down the 7/2/50MH or 7/2/50MH@BMP-2 adhesive as the bone phase, followed with application of 7/2 or 7/2@TGF-2 adhesive as the cartilage phase. We took macroscopical photographs to evaluate the cartilage regeneration at 8- and 12-week post-surgery. We observed neo-cartilage tissue formation at the implantation sites in the 7/2, BL, and BL/GFs groups at 8-week post-surgery (FIG. 6A). The neo-cartilage was observed to cover most of the defect in BL/GFs groups at 8-week post-surgery. After 12 weeks, complete repair of the defect site with tissue resembling the native cartilage with excellent integration with the implant was observed in the BL/GFs group. The blank and 7/2/50MH groups showed discontinuous and incomplete integration at the defect site (FIG. 6A, bottom). Moreover, we quantitatively analysed the performance of the adhesives by calculating the International Cartilage Repair Society (ICRS) macroscopic repair score. The BL/GFs group scored 2.36 and 3.16 folds higher than the 7/2 and 7/2/50MH groups, respectively (FIG. 6C). We then performed micro-CT analysis to evaluate the regeneration of subchondral bone. 7/2/50MH, BL and BL/GFs groups showed large quantities of calcified new bone tissues formed in the subchondral layer after 8 weeks (FIG. 6B), whereas the blank and the 7/2 groups showed a relatively sparse bone connection with minimal evidence of bone repair throughout. The BL/GFs group demonstrated highest bone mineral density (BMD) and bone volume/total tissue volume (BV/TV) compared to other groups (FIGS. 6C and D).

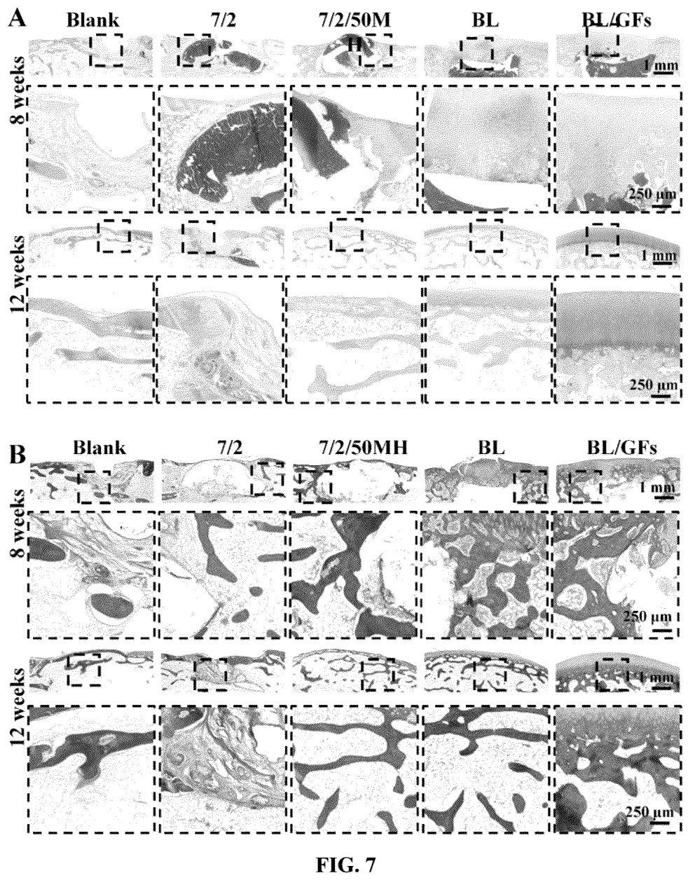

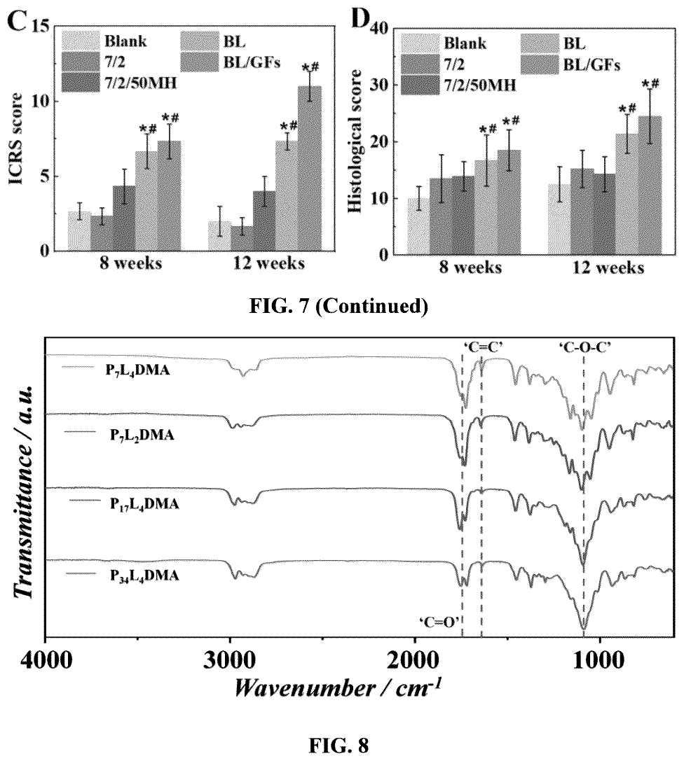

After macroscopic evaluation, we proceeded to evaluate the chondrogenesis and osteogenesis in vivo through Safranin-O/Fast-Green (FIG. 7A) and Masson trichrome staining (FIG. 7B). At week 8, BL and BL/GFs groups demonstrated an overall superior performance in cartilage repair, with the defect sites filled with newly developed cartilage tissues with the rich glycosaminoglycans (GAGs) (FIG. 7A). The 7/2 and 7/2/50MH groups showed slightly increased GAG deposition while the blank group showed minimal cartilage tissues. At week 12, the BL/GFs group demonstrated better chondroprotective effects with more complete and thicker cartilage than other groups, indicating excellent integration with the surrounding tissues (FIG. 7A). Similarly, the BL/GFs group presented the best cartilage regeneration efficacy in histological scoring, indicating the TGF-β1 further improved the cartilage repair process (FIG. 7C). In Masson trichrome staining for osteogenesis, we found BL/GFs demonstrated the best subchondral bone repair at week 12, with significantly enhanced neobone formation with more dense subchondral tissues and better integration with the host tissues (FIG. 7B). Additionally, the quantification of the red staining, which indicated the mature bone formation, showed that the BL/GFs group embodied the maturation of bone tissues (FIG. 7D). Altogether, these results confirmed the superior performance of our injectable bilayered adhesives for in vivo OC defect repair. The BL/GFs adhesives induced excellent repair and integration in both cartilage and subchondral layer through simultaneously modulating the physical (differential stiffness between layers) and biological (incorporation of TGF-β1 and BMP-2) microenvironment at the defect site.

In this study, we developed injectable hydrophobic adhesives that can crosslink rapidly in a subaqueous environment, forming an anisotropic structure with strong interlayer bonding that simultaneously modulate mechanical-biochemical cues for enhancing OC repair. We use the liquid hydrophobic photocrosslinkable poly(lactide-co-propylene glycol-co-lactide) dimethacrylates (PₘLₙDMA) as the cartilage phase and adopt polymer encapsulating methacrylated hydroxyapatite nanoparticles (PₘLₙDMA/MH) as the mineralized subchondral bone phase. Due to the hydrophobic nature of the materials, we find the two phases can be confined within the defect site without dispersing among the body fluid and can be solidified within 180 second to form the bi-layered scaffolds. The presence of olefins in the PₘLₙDMA and the PₘLₙDMA/MH enables the two layers to form strong covalent bonding at the interface. Through the systematical modulation of the adhesives' composition, we can match the mechanical properties of the two phases with the native OC tissues. Additionally, we demonstrated the adhesives' ability in encapsulating and releasing the chondrogenic TGF-β1 and osteogenic BMP-2 for over 60 days in further stimulating regeneration. Notably, we observed synergistic effect of the mechanical cues and the growth factors in directing the cellular functions of MSCs towards chondrogenesis and osteogenesis in vitro and in vivo via mediating the crosstalk between the mechanotransduction and the growth factor signaling pathways.

Our laminous adhesives can be directly injected and rapidly solidified in situ under wet conditions to support OC healing, preventing injection spillage and minimizing the local tissue damage and treatment time needed for measurements in tailor-made joint operations. Our adhesives modulate the cartilage regeneration through synergistically activating the TGF-β/Smad and Hippo-YAP/TAZ signaling pathways, while enhance the bone regeneration through synergistically activating the FAK and PI3K-Akt signaling pathways. Additionally, all components used in our adhesives are metabolizable without any extraction of living cells or tissues, bypassing ethical concerns which may hinder clinical translation. These traits and the practicality of our underwater-crosslinking liquid hydrophobic materials will prove to be attractive to hospitals and enterprises interested in translation of our research. Altogether, this research work is highly translational and will excel in clinical trials, ultimately benefiting patients who suffer from OC fracture, osteoarthritis and osteoporosis, relieving burden from families and society.

Bio-Inspired Surface Texture on PₘLₙDMA

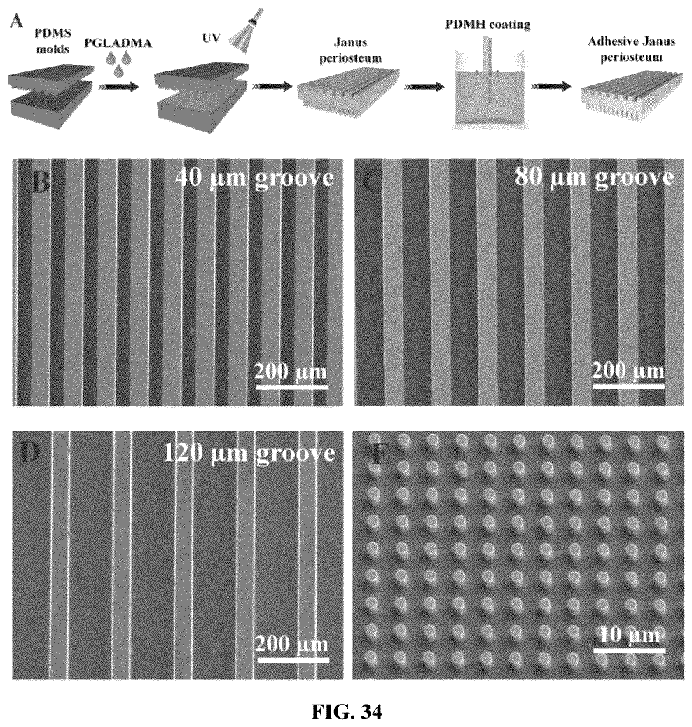

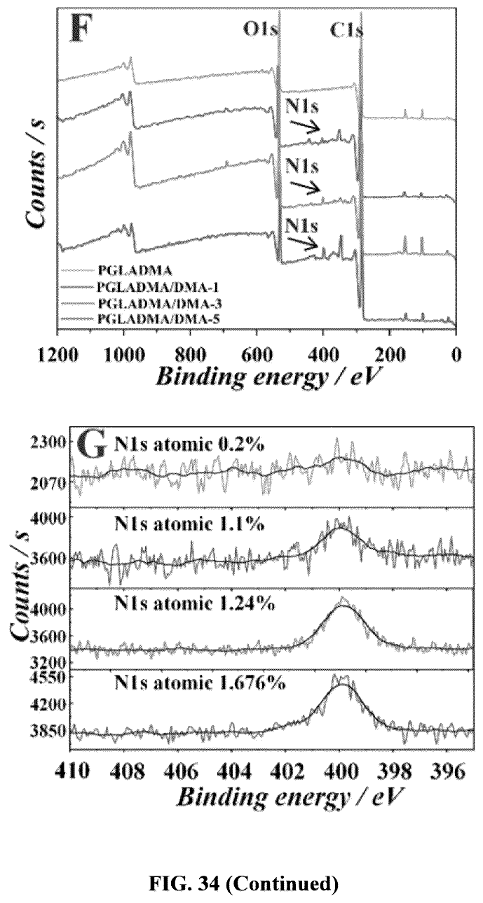

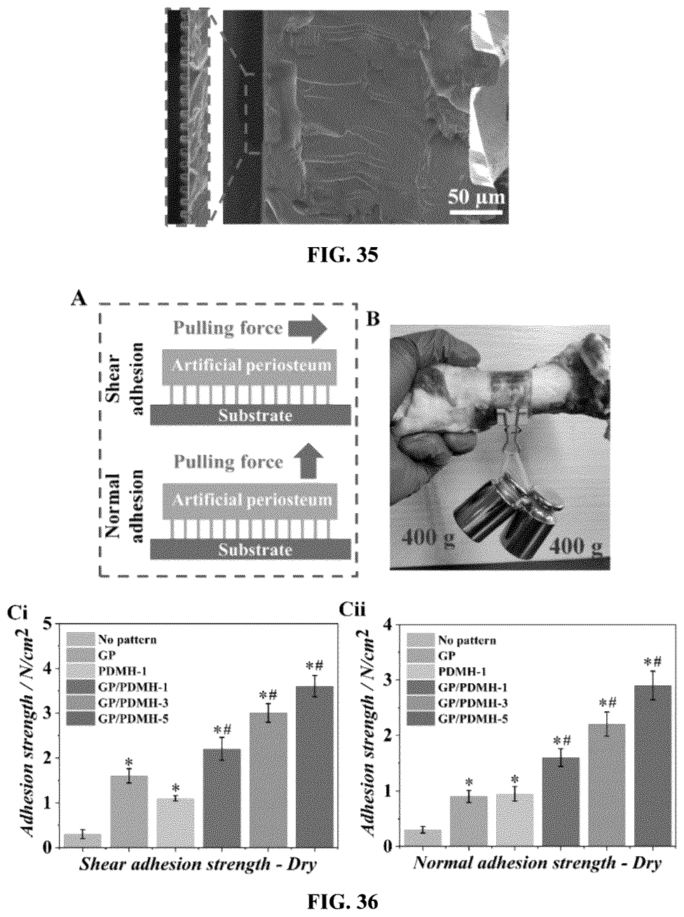

An adhesive Janus periosteum made of PₘLₙDMA was fabricated using photo-crosslinking-assisted injection molding and poly(dopamine methacrylamide-co-hydroxyethyl methacrylate) (PDMH) dip-coating (FIG. 34A). To evaluate the structure integrity of the fabricated adhesive Janus periosteum, we examined the morphology of the PₘLₙDMA-PDMH surfaces using scanning electron microscopy (SEM). We found that the exterior of the adhesive Janus periosteum was carved with precise microgrooves at 40 μm, 80 μm or 120 μm intervals (FIGS. 34B-D) and the other surface was covered with gecko feet pad like fibrillar setae arrays (2 μm in diameter and 4 μm in height) (FIG. 34E). Such high-resolution structure could be attributed to the excellent moldability of the PₘLₙDMA polymer and indicated that the coating process has no detrimental effect on the surface structure. Besides, the cross-sectioned images further demonstrated the anisotropic nature of the adhesive Janus periosteum (FIG. 35).

To evaluate the success immobilization of PDMH on the PₘLₙDMA surfaces, we then investigated the chemical compositions of the PₘLₙDMA surfaces using X-ray photoelectron spectroscopy (XPS) (FIG. 34F). The appearance of the N 1s at about 400 eV could be observed in all PDMH coating samples and with the increase of the PDMH concentration from 1 mg/mL to 5 mg/mL, the signal intensity of the N is increased. These results confirmed that the PDMH could be immobilized onto the PₘLₙDMA surfaces, and the coating amount could be simply modulated by the PDMH concentration, which enables the precise modulation of adhesive strength by PDMH coating.

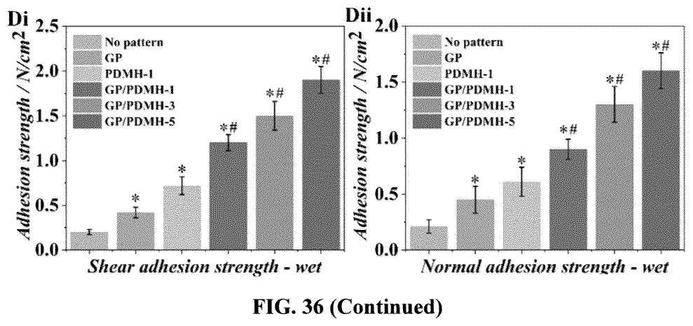

On the account of the successful fabrication of the adhesive Janus periosteum, we then moved to the evaluation of the adhesiveness of the periosteum. Ideally, the artificial periosteum should stably adhere to the defect site and withstand the pressure from the body fluids in both shear and normal directions to avoid rupture or detachment post-implantation. Such adhesiveness could therefore inhibit the possible osteolytic zone and bone resorption. The shear and normal tissue adhesion strength of our periosteum in dry and wet conditions were assessed as illustrated in FIG. 36A. Under dry condition, we found that either the gecko inspired pattern (GP) or the mussel inspired adhesive PDMH coating could significantly increase the shear and normal adhesion strength due to the exploitation of Van der Waals or the catechol-rich surface chemical structure. The strongest adhesion could be achieved when both designs were combined because of the coordinated interfacial friction and bonding. The shear and normal adhesion strength of the GP-PDMH-5 group exhibited 3.6 N/cm² and 2.8 N/cm², which was comparable with the previous studies (FIG. 36C). To provide an index for the application of the Janus periosteum under physiological wet condition, we also evaluated the shear and normal adhesion strength under water. As expected, the PDMH coating exhibited improved water-resistant capacity compared to the GP structures; however, it was still observed that the combination of the GP structures and the PDMH coating could generate stronger adhesion underwater as the GP/PDMH-5 group presented 1.9 and 1.6 N/cm² of the shear and normal adhesion strength (FIG. 36D). Additionally, the application of the adhesive Janus periosteum to fresh bone could withstand 800 g weight (FIG. 36B). These results demonstrated our adhesive Janus periosteum embodied robust dry and wet adhesion in their respective applications.

### Experimental

Synthesis and characterization of PₘLₙDMA and MH: Different formulations of PₘLₙDMA (‘m’ and ‘n’ referred to the unit length of PPG and molar ratio of LA respectively) were synthesized. Take P₇L₂DMA as an example: we catalyzed the reaction between 20 g PPG (Mw=425, Sigma-Aldrich, China) and 13.5 g LA (Sigma-Aldrich, China) using stannous octoate for 6 hours at 150° C., under argon protection through a ring-opening polymerization reaction. 2.11 g of methacryloyl chloride (MAC; Macklin, China) and 2.0 g trimethylamine (TEA; Macklin, China) were then diluted in dichloromethane (DCM; Duksan, China) and added to the product in ice bath. Next, the products were extracted by dissolving in 100 mL of diethyl ether followed by vacuum filtration and rotary evaporation. An attenuated total reflectance Fourier transform infrared (ATR-FTIR, Thermo, US) were used to identify specific chemical bonding of the final product.

Hydroxyethyl methacrylate (HEMA; Sigma, China) was used to graft the reactive methacrylate groups to the surface of nano-hydroxyapatite (nHA, 80-100 nm, Macklin, China). Briefly, 20 g nHA was reacted with 4 mL hexamethylene diisocyanate (HDI; Sigma-Aldrich, China) at 50° C. for 24 hours under the catalysis of dibutyl-tindilaurate (Macklin Reagent, China). 8 mL HEMA was then added to graft the methacrylate groups to nHA, and the reaction was kept for 5 hours. The final product MH was thoroughly washed with DCM. The ATR-FTIR was adopted to verify the coupling of the HEMA chain to the nHA surface. The grafting efficiency was calculated by the thermogravimetric analyzer (TGA, Mettler Toledo, USA).^{[26]}

Incorporation of growth factors into the adhesives: We incorporated TGF-β1 (Peprotech, China) and BMP-2 (Peprotech, China) into the prepared P₇L₂DMA and P₇L₂DMA/50MH adhesives, respectively. For TGF-β1 loaded P₇L₂DMA (7/2@TGF-β1), 1.5 μg TGF-β1 dry powder was well mixed with 500 mg adhesives. Then, we cast the adhesives into a disk with a diameter of 10 mm and used a UV lamp to crosslink the adhesives. The crosslinked samples were incubated in 10 mL PBS at room temperature. To analyze the amount of TGF-β1 released at predetermined time points, we collected 1 mL PBS from the incubation medium at a specific time interval, and analyzed the extract using the TGF-β1 ELISA Kit (Bioss, China).^{[16b]} After each extraction, we replaced the incubation medium with the same volume of fresh PBS. The BMP-2 loaded P₇L₂DMA/50MH (7/2/50MH@BMP-2) adhesives were prepared similarly, and the release of BMP-2 was characterized using BMP-2 ELISA Kit (Bioss, China).

Preparation and characterization of cartilage and subchondral bone adhesives: The photocrosslinkable PₘLₙDMA was prepared by mixing the PₘLₙDMA (90 wt %) with additional HEMA (9 wt %) and Irgacure® 819 (1 wt %). The PₘLₙDMA/MH nanocomposites were prepared by mixing the PₘLₙDMA with various MH concentrations (e.g., 10, 30, and 50 wt. %). The injectability of the adhesives was characterized using a rheometer (Anton Paar, Austria) at room temperature. The parallel plate was 50 mm diameter with 1 mm gap width. The viscosity parameter was set as the rotational test mode and the shear rate ranged from 1 to 100 s⁻¹. Synthesized materials were then injected into the water to evaluate the underwater injectability and shape integrity of the adhesives. The photocrosslinking kinetic of the adhesives was characterized using FTIR^{[7]} Briefly, after different UV irradiation time, the changes of the normalized 1640 cm⁻¹ peak intensity owing to the ‘C═C’ was recorded, and the polymerization ratio (Rₚ) was calculated using the following equation:

$${R}_{p} ( \% ) = \frac{{H}_{0} - {H}_{1}}{{H}_{0}} \times 100 \% ,$$

where the H₀ is the initial normalized intensity of ‘C═C’, and H₁ is the normalized intensity of ‘C═C’ after photocrosslinking at a predetermined time point. At the same time, a thermal camera (FLIR, US) was used to record the change in temperature during the photocrosslinking process. Afterwards, compressive and tensile mechanical properties of the materials photocrosslinked in air or underwater were tested following the ATSM 695 and D638 standards, respectively.^{[27]} For the compressive test, the adhesives were injected into a Teflon mold (Ø6 mm×3 mm) in air or underwater, followed by UV irradiation for 200 s. Then, the samples were compressed with 1 mm/min speed until a fracture was observed. We tested the tensile properties under the same conditions, but instead, we exposed the material to tensile tension until a visible break.

Preparation and characterization of the bi-layered osteochondral scaffolds: The anisotropic bi-layered OC scaffolds were prepared by topping the PₘLₙDMA layer onto the PₘLₙDMA/MH layer followed by photocrosslinking with UV light for 200 s. Different shapes of Teflon molds (round, moon, triangle and star) were prepared to evaluate the injectability of the adhesives. The interfacial morphology was observed by SEM (Tescan VEGA3, Czech), and the interfacial bonding strength was evaluated by lap shear test based on the ASTM D3528-96 standard. Briefly, for the lap shear test, the PₘLₙDMA or PₘLₙDMA/MH was injected into a 20 mm×20 mm Teflon mold followed by photocrosslinking to form bi-layered samples. Then, the bi-layered samples were bonded to a glass slide via a 3M scotch tape to evaluate the shear strength. The bi-layered adhesives photocrosslinked or fixed with commercial glue (ethyl cyanoacrylate, Loctite, USA) were set as controls. The normal force and shear force were both performed. The separation speed was set at 1 cm/min, and we used the critical peel separation strength to determine the adhesive force of the bonding interface.

Biocompatibility evaluation of the adhesives: Disc-like adhesives samples (Ø10 mm×1 mm) of 7/2, 7/2@TGF-β1, 7/2/50MH, and 7/2/50MH@BMP-2 were first prepared. The biocompatibility of the adhesives was evaluated by seeding rBMSCs (Cyagen, China) with a density of 1×10⁴ cells/cm² onto the adhesives. The cell culture medium was prepared by Minimum Essential Medium α (α MEM, Gibco, China) supplemented with 10% fetal bovine serum (Gibco, China) and 1% penicillin/streptomycin (Gibco, China). We assessed the cell viability and proliferation on 1, 2, and 3 days post-incubation using Live/Dead kit and Picogreen® DNA quantification assay (Thermo Fisher, Hong Kong). Cell viability was quantified using the ratio of viable cells to all cells in six randomly selected images. Cell proliferation was tested after culture for 1, 2, and 3 days via the Picogreen Assay. In addition, F-actin and DAPI staining were performed to assess the cell morphology. After one day of culture, cells were fixed with 4% paraformaldehyde for 15 minutes, permeabilized for 15 minutes in blocking solution (Bioshark, China), and incubated for 30 minutes with Alexa 488 phalloidin (Thermo Fisher, Hong Kong). DAPI (4′,6-diamidino-2-phenylindole, Thermo Fisher, Hong Kong) was used to stain the nuclei. Six randomly selected images were chosen for cell spreading area measurement using Image J software.^{[28]}

rBMSCs' chondrogenic and osteogenic differentiation analysis: To study the effect of adhesives on chondrogenic differentiation of rBMSCs, we seeded 4×10⁴ cells/cm² of rBMSCs on 7/2/50MH, 7/2, and 7/2@TGF-β1 samples (Ø10 mm×1 mm) and cultured with chondrogenic medium supplemented with 100 μg/mL sodium pyruvate (Sigma-Aldrich, China), 100 nM dexamethasone (Sigma-Aldrich, China), 1% insulin-transferrin-selenium (ITS; Sigma-Aldrich, China) and 50 μg/mL ascorbic acid (Sigma-Aldrich, China). rBMSCs were fixed on day 3 and day 7 with paraformaldehyde for 10 min followed by staining with Safranin O staining solution (Solarbio, China) for 5 min. On day 7 and 14, the Alcian blue staining (Solarbio, China) was applied with similar methods to further evaluate the chondrogenesis of rBMSCs. All images were taken by an optical microscope (Nikon, Japan), and the positive staining area was quantified using Image J software.

For studying the effect of adhesives on osteogenic differentiation of rBMSCs, 4×10⁴ cells/cm² of rBMSCs were seeded on 7/2, 7/2/50MH, and 7/2/50MH@BMP-2 samples (Ø10 mm×1 mm) and cultured with osteogenic medium (Cyagen, China). The ALP activity of the seeded rBMSCs on day 3 and 7 of incubation was studied by fixing the cells with paraformaldehyde for 10 min, followed by staining with the BCIP/NBT solution (Beyotime, China).^{[29]} The ALP activity of the rBMSCs was quantified using the ALP activity kit (Beyotime, China) with total protein normalization.^{[29]} The ARS staining was used to study the mineralization of the seeded rBMSCs. After cell fixation, the ARS staining solution (Solarbio, China) was added to the cell-seeded adhesives for 20 min. Then, the sample was gently washed with deionized water until the color disappeared. All images were taken using an optical microscope. 10% cetylpyridinium chloride (CPC, Sigma, USA) solution was added to dissolve the ARS stain for 3 hours at room temperature. ARS staining was then quantified using a Microplate reader (BioTek, US) by measuring the absorbance of the ARS extracts at 592 nm. ^{[30]} Quantitative reverse transcription-polymerase chain reaction (qRT-PCR) was used to measure the gene expressions of chondrogenesis and osteogenesis-related markers (collagen type II (Col-2), aggrecan, Runt-related transcription factor 2 (Runx2), collagen type I (Col-1) on day 3 and 7 of incubation. Briefly, 2×10⁴ cells/cm² of rBMSCs were seeded onto two sets of adhesives for culturing. Total RNA Kit (Omega, Hong Kong) was used to isolate the total RNA from cells cultured on adhesives on day 3 and 7 for analysis. After generating the cDNA by reverse transcription, we performed the qRT-PCR by CFX 96 detection system (Bio-rad, USA). The primer sequences of target genes are listed in FIG. 32.

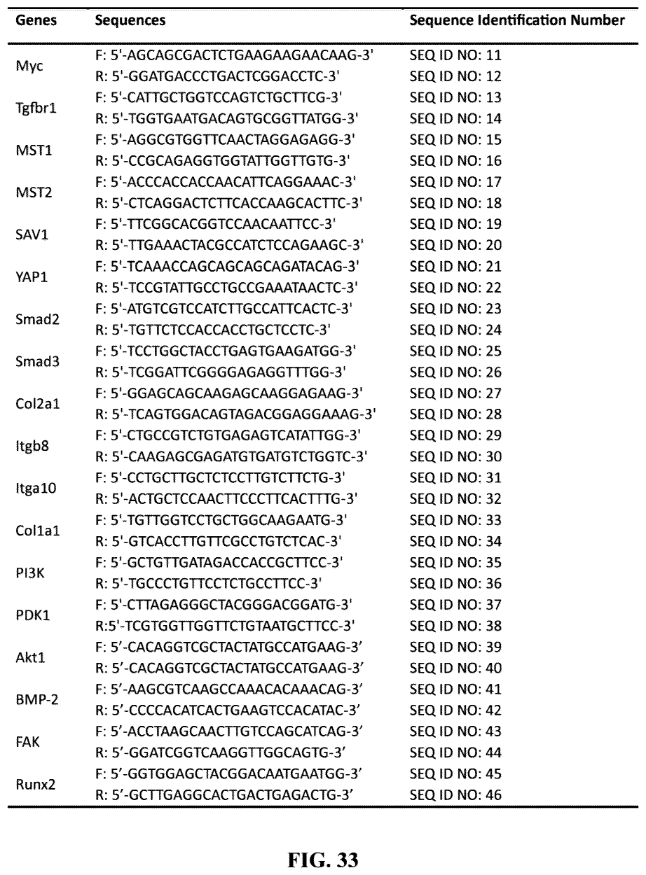

Bioinformatic analysis of the biophysical and biochemical stimulated cell differentiation: 4×10⁴ cells/cm² of rBMSCs were seeded onto two different sets of adhesives for 3 days. The first set comprised of 7/2/50MH and 7/2@TGF-β1 adhesives to examine the effect of soft ECM and TGF-β1. The second set of adhesives comprised of 7/2 and 7/2/50MH@BMP-2 to examine the effect of stiff ECM and BMP-2. Then, rBMSCs were lysed by the TRIzol reagent (Invitrogen, USA). RNA sequencing was performed using an Illumina Novaseq™ 6000 platform (LC-Bio Technology CO., Ltd., China) following the manufacturer's protocol. The “edgeR” R package was used to analyze the differentially expressed genes (DEGs) using pair-wise comparisons, and differentially expressed mRNAs were selected based on a fold change>2 or <0.5. GO analysis and KEGG enrichment were performed using the OmicStudio tools (https://www.omicstudio.cn/tool). The primer sequences of the target genes are listed in FIG. 33.

In vivo biological characterizations of bi-layered osteochondral scaffolds: All animal evaluation was performed with approval from the Ethics Committee of the Hong Kong Polytechnic University (21-22/125-BME-R-GRF). 60 New Zealand white rabbits (male, weight from 3.0 to 3.5 kg) were randomly divided into five groups: blank group, 7/2 group (filled with P₇L₂DMA adhesives), 7/2/50MH group (filled with P₇L₂DMA/50% MH adhesives), BL group (filled with bi-layered adhesives) and BL/GFs group (filled with bi-layered adhesives with TGF-β1 and BMP-2 incorporation in the different layers). A full-thickness osteochondral cylindrical defect (5-mm diameter and 4-mm depth) was introduced on the trochlear groove of the rabbits' distal femur using a dental drill.^{[31]} The defects were then filled with the assigned adhesives, followed by UV irradiation for 200 s. The operated knee joints were closed with 4-0 sutures and intramuscular antibiotic adoption. After 8- and 12-week of implantation, we sacrificed the rabbits with CO₂ suffocation and harvested the femur samples. The quality of OC repair was assessed by two trained and blinded observation according to the ICRS scores system. Micro-CT scanning (SkyScan 1176, Belgium) was additionally performed to evaluate the subchondral bone regeneration. The scanning parameters were set as source voltage 100 kV, source current 200 μA and Al+Cu filter. Threshold from 45 to 255 were set to reconstruct the bone morphology. CTAn software was used to perform 3D reconstruction and quantification of BMD and BV/TV. After micro-CT analysis, the histology analysis using Safranin O and Masson trichrome staining was performed to study the cartilage repair and new bone formation in the defect sites. Histological repair score was calculated following a modified method in the previous studies.^{[32]}

### Bio-Inspired Surface Textures

Synthesis of PₘLₙDMA: Photo-crosslinkable PₘLₙDMA polymer was synthesized by a two-step reaction. Briefly, 40 g PPG and 23 g LA were mixed together and reacted for 6 hours at 150° C. under nitrogen protection with stannous octoate as catalyst to obtain the PGLA. Then, methacrylate moieties were grafted to the PGLA chain by incorporation of 4.22 g methacryloyl chloride at 0° C., both of which were diluted in dichloromethane. The product was purified by vacuum filtration after dissolving in diethyl ether and collected after rotary evaporation of the residual solvent. The attenuated total reflectance Fourier transform infrared (ATR-FTIR) (Bruker, US) was adopted to characterize the synthesized PₘLₙDMA.

Synthesis of PDMH: The PDMH was synthesized as follows. Briefly, the inhibitor of HEMA was first removed by basic alumina. Then, 1.7 mL HEMA, 0.62 g dopamine methacrylamide (DMA) and 42 mg azobisisobutyronitrile were added to a round flask with 10 mL DMF. The mixture was bubbled with nitrogen for 30 minutes followed by co-polymerization at 60° C. The resulting mixture was diluted by 10 mL methanol followed by dropwise adding into 150 mL diethyl ether to precipitate the synthesized PDMH copolymer. The purified co-polymer was thoroughly dried in a vacuum oven. ATR-FTIR was used to characterize the synthesized PDMH copolymer.

Fabrication of the anisotropic periosteum: The gecko-inspired and natural periosteum mimicking polydimethylsiloxane (PDMS) negative molds were fabricated using standard microfabrication techniques by Nanzhi Institute of Advanced Optoelectronic Integration (Nanjing, China). The PₘLₙDMA precrosslinking solution was prepared by thoroughly mixing 90 wt. % PₘLₙDMA, 9 wt. % HEMA and 1 wt. % Irgacure® 819 by the homogenizer. Then, the precrosslinked PₘLₙDMA polymer was first placed between two PDMS negative molds with 250 μm interval. One PDMS mold was carved with fibrillar arrays (2 μm in diameter and 4 μm in height) and the other was carved with microgrooved patterns (40, 80, 120 μm). The PₘLₙDMA was then treated with vacuum to remove the trapped air, followed by photo-crosslinking with a UV lamp (365 nm, 5 mW/cm²) for 200 seconds to fully crosslink the P PₘLₙDMA. For the PDMH coated Janus periosteum, the PDMH was first dissolved in the ethanol with 1, 3, 5 mg/mL concentration. Then, the prepared naked Janus periosteum was immersed in different concentration of the PDMH/ethanol solution for 2 hours followed by the vacuum dry overnight at room temperature.

Tissue adhesion characterization of anisotropic periosteum: Both shear and normal tissue adhesion tests under dry and wet condition were performed based on previous protocol.^{[14]} Briefly, a 10 mm×10 mm porcine sausage skin membrane was glued to the glass slide and the other side of the porcine sausage skin was used for the adhesion test. Then, the fabricated anisotropic periosteum was cut into 10 mm×10 mm and also glued to the glass slide. The surface with the gecko inspired pattern was used for the adhesion test. The shear and normal adhesion tests under dry condition were first performed. In the shear adhesion test, the prepared glass slides were placed together in the 10 mm×10 mm area compressed with a constant 10 N force to establish a good contact with each other. Then, the two glass slides were pulled in parallel to the glass surface at a constant rate of 1 mm/min to measure the shear tissue adhesion strength. In the normal adhesion test, the tightly contacted glass slides were pulled perpendicularly to the glass surface at a constant rate of 1 mm/min to measure the normal tissue adhesion strength. To further characterize the tissue adhesion behavior under wet condition, we also performed the wet adhesion test in which the two prepared slides were contacted and compressed under water. Then, the shear and normal tissue adhesion were measured following the previous protocol.

Statistical analysis: All tests were performed in quadruplicate unless otherwise indicated and the values were presented as mean±standard deviation. One-way or two-way ANOVA by Tukey post-hoc was adopted to conduct statistical evaluation between each group with GraphPad Prism Software (GraphPad Software Inc.). A difference at *p<0.05 and ^{#}p<0.05 was considered statistically significant.

What is claimed is:

1. A multi-layered scaffold comprising a cartilage phase layer disposed on a surface of a bone phase layer, wherein the multi-layered scaffold is prepared by photopolymerizing a multi-layer scaffold precursor comprising: a first layer disposed on a surface of a second layer, wherein the first layer comprises a first tri-block poly(lactide-co-propylene glycol-co-lactide) dimethacrylate; and the second layer comprises a second tri-block poly(lactide-co-propylene glycol-co-lactide) dimethacrylate, nanoparticles covalently conjugated to one or more hydroxyethyl methacrylate moieties via an optional linker, and a photoinitiator, wherein the nanoparticles comprise hydroxyapatite, tricalcium phosphate, silicon dioxide, bioglass, or a mixture thereof.

2. The multi-layered scaffold of claim 1, wherein each of the first tri-block poly(lactide-co-propylene glycol-co-lactide) dimethacrylate and the second tri-block poly(lactide-co-propylene glycol-co-lactide) dimethacrylate independently comprises polypropylene glycolide having the structure —[O(CHMe)(CH₂)]ₘO—, wherein m is 2-40.

3. The multi-layered scaffold of claim 1, wherein each of the first tri-block poly(lactide-co-propylene glycol-co-lactide) dimethacrylate and the second tri-block poly(lactide-co-propylene glycol-co-lactide) dimethacrylate independently comprise polylactide and polypropylene glycolide in a molar ratio between 1-5 to 1, respectively.

4. The multi-layered scaffold of claim 1, wherein the second layer comprises hydroxyapatite nanoparticles covalently conjugated to one or more hydroxyethyl methacrylate moieties via an optional linker at a concentration of 10-70% wt/wt relative to the total weight of the second tri-block poly(lactide-co-propylene glycol-co-lactide) dimethacrylate and hydroxyapatite nanoparticles covalently conjugated to one or more hydroxyethyl methacrylate moieties via an optional linker.

5. The multi-layered scaffold of claim 1, wherein each of first layer and the second layer independently further comprises one or more bioactive factors.

6. The multi-layered scaffold of claim 5, wherein the one or more bioactive factors are selected from the group consisting of a nucleic acid, a protein, a peptide, a cytokine, a hormone, a cell, and a growth factor.

7. The multi-layered scaffold of claim 1, wherein the first layer further comprises TGF-β1; and the second layer further comprises BMP-2

8. The multi-layered scaffold of claim 1, wherein the hydroxyapatite nanoparticles are covalently conjugated to one or more hydroxyethyl methacrylate moieties via a diisocyanate linker.

9. The multi-layered scaffold of claim 8, wherein the diisocyanate linker is OCN(CH₂)ₙNCO, wherein n is a whole number selected from 2-8.

10. The multi-layered scaffold of claim 1, wherein the photoinitiator is photoinitiator is an acylphosphine oxide.

11. The multi-layered scaffold of claim 1, wherein at least one exterior surface of the multi-layered scaffold comprises microgrooves, micropillars, or a combination thereof.

12. The multi-layered scaffold of claim 1, wherein an exterior surface of the cartilage phase layer comprises microgrooves and an exterior surface of the bone phase layer comprises micropillars.

13. The multi-layer scaffolded of claim 1, wherein the step of photopolymerizing comprises photopolymerizing the first layer and the second layer simultaneously or photopolymerizing the second layer and then photopolymerizing the first layer.

14. The multi-layer scaffolded of claim 1, wherein
each of the first tri-block poly(lactide-co-propylene glycol-co-lactide) dimethacrylate and the second tri-block poly(lactide-co-propylene glycol-co-lactide) dimethacrylate independently comprises polypropylene glycolide having the structure —[O(CHMe)(CH₂)]ₘO—, wherein m is 7-34;
each of the first tri-block poly(lactide-co-propylene glycol-co-lactide) dimethacrylate and the second tri-block poly(lactide-co-propylene glycol-co-lactide) dimethacrylate independently comprise polylactide and polypropylene glycolide in a molar ratio between 2-4 to 1, respectively;
the hydroxyapatite nanoparticles are covalently conjugated to one or more hydroxyethyl methacrylate moieties via a diisocyanate having the structure: OCN(CH₂)ₙNCO, wherein n is a whole number selected from 4-8;
the second layer comprises the hydroxyapatite nanoparticles covalently conjugated to one or more hydroxyethyl methacrylate moieties via the diisocyanate linker at a concentration of 10-50% wt/wt relative to the total weight of the second tri-block poly(lactide-co-propylene glycol-co-lactide) dimethacrylate and the hydroxyapatite nanoparticles covalently conjugated to one or more hydroxyethyl methacrylate moieties via an optional linker; and
each of the first layer and the second layer optionally independently further comprises one or more bioactive factors.

15. The multi-layer scaffolded of claim 1, wherein each of the first tri-block poly(lactide-co-propylene glycol-co-lactide) dimethacrylate and the second tri-block poly(lactide-co-propylene glycol-co-lactide) dimethacrylate independently comprises polypropylene glycolide having the structure —[O(CHMe)(CH₂)]ₘO—, wherein m is 7;
each of the first tri-block poly(lactide-co-propylene glycol-co-lactide) dimethacrylate and the second tri-block poly(lactide-co-propylene glycol-co-lactide) dimethacrylate independently comprise polylactide and polypropylene glycolide in a molar ratio of 2 to 1, respectively;
the hydroxyapatite nanoparticles are covalently conjugated to one or more hydroxyethyl methacrylate moieties via a diisocyanate linker having the structure: OCN(CH₂)₆NCO; and
the second layer comprises the hydroxyapatite nanoparticles covalently conjugated to one or more hydroxyethyl methacrylate moieties via the diisocyanate linker at a concentration of 50% wt/wt relative to the total weight of the second tri-block poly(lactide-co-propylene glycol-co-lactide) dimethacrylate and the hydroxyapatite nanoparticles covalently conjugated to one or more hydroxyethyl methacrylate moieties via the diisocyanate linker.

16. The multi-layer scaffolded of claim 15, wherein the first layer further comprises TGF-β1; and the second layer further comprises BMP-2.

17. A method of fabricating the multi-layer scaffolded of claim 1, the method comprising:
providing a first layer precursor;
providing a second layer precursor; and
photopolymerizing the first layer precursor and the second layer precursor thereby forming the multi-layer scaffolded,
wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof.

18. A method of fabricating the multi-layer scaffolded of claim 1, the method comprising:
depositing a second layer precursor on a substrate thereby forming the second layer;
depositing a first layer precursor on a surface of the second layer thereby forming the multi-layer scaffold precursor; and
photopolymerizing the multi-layer scaffold precursor thereby forming the multi-layer scaffolded,
wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof.

19. A method of repairing an osteochondral complex (OC) defect in a subject in need thereof, the method comprising: depositing a second layer precursor at the OC defect site thereby forming a second layer;
depositing a first layer precursor on a surface of the second layer thereby forming the multi-layer scaffold precursor; and
photopolymerizing the multi-layer scaffold precursor thereby forming the multi-layer scaffolded of claim 1,
wherein the first layer precursor comprises: the first tri-block poly(lactide-co-propylene glycol-co-lactide) dimethacrylate; and the second layer precursor comprises the second tri-block poly(lactide-co-propylene glycol-co-lactide) dimethacrylate, nanoparticles covalently conjugated to the one or more hydroxyethyl methacrylate moieties via an optional linker, and the photoinitiator, wherein the nanoparticles comprise hydroxyapatite, tricalcium phosphate, silicon dioxide, bioglass, or a mixture thereof.
